(19) United States
(12) Patent Application Publication
(10) Pub. No.: US 2021/0340113 A1
(43) Pub. Date: Nov. 4, 2021

(54) PROCESS FOR PREPARATION OF OPTICALLY ENRICHED ALDOL COMPOUNDS

(71) Applicant: BASF SE, Ludwigshafen (DE)

(72) Inventors: Karsten Koerber, Ludwigshafen (DE); Nikolas Huwyler, Ludwigshafen (DE); Arun Narine, Ludwigshafen (DE); Martin John McLaughlin, Ludwigshafen (DE); Kailaskumar Borate, Mumbai (IN)

(21) Appl. No.: 17/284,199

(22) PCT Filed: Oct. 18, 2019
(86) PCT No.: PCT/EP2019/078309

§ 371 Date: Apr. 9, 2021

(30) Foreign Application Priority Data
Oct. 29, 2018 (EP) 18203033.8

(51) Int. Cl. C07D 261/04; C07D 491/107

(52) U.S. Cl. CPC C07D 261/04; C07D 491/107

(57) ABSTRACT

The present invention relates to a process for preparing benzylic amides of formula (I) wherein the variables are as defined in the specification, and the shown enantiomer has at least 50% ee; by condensation of a ketone o formula (II) with an acetyl compound of formula (III) in the presence of a catalyst of formula (IV) wherein the variables are as defined in the specification.

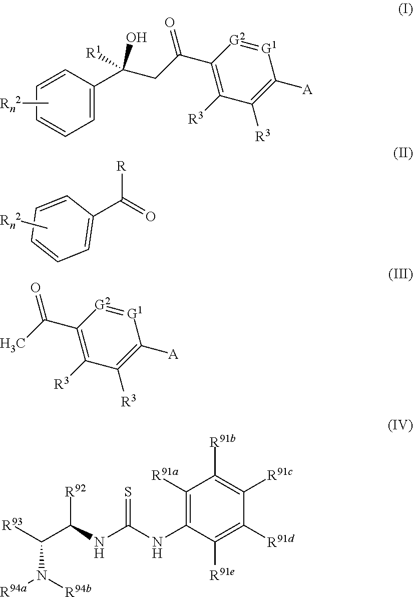

The present invention relates to a process for the preparation of optically enriched aldol compounds of formula I

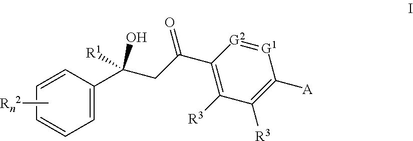

wherein
R¹ is halomethyl;
each R² is independently H, halogen, CN, N₃, NO₂, SCN, SF₅, C₁-C₆-alkyl, C₃-C₈-cycloalkyl, C₂-C₆-alkenyl, C₂-C₆-alkynyl, which groups are unsubstituted, partially or fully halogenated and/or substituted with one or more same or different R⁸,
   Si(R¹²)₃, OR⁹, S(O)ₙR⁹, NR^{10a}R^{10b},
   phenyl which is unsubstituted or partially or fully substituted with R¹¹, and a 3- to 10-membered saturated, partially or fully unsaturated heteromonocyclic or heterobicyclic ring containing 1, 2, 3 or 4 heteroatoms N, O, and/or S as ring members, which ring is unsubstituted, or substituted with one or more same or different R¹¹, preferably the unsubstituted or substituted HET;
n is 0, 1, or 2;
G¹, G² are each CR³, or together form a sulfur atom;
each R³ is independently selected from the meanings mentioned for R², or two R³ bonded to adjacent carbon atoms may form a five- or sixmembered saturated, partially or fully unsaturated carbocyclic ring, or a dihydrofurane, or
R³ bonded to carbon atom in position G¹ form a bond to the chain *-Q-Z— in group A²;
A is a group A¹, A², A³, or A⁴; wherein
   A¹ is C(═W)Y;
      W is O, or S;
      Y is N(R⁵)R⁶, or OR⁹;
   A² is

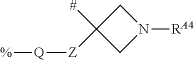

wherein # denotes the bond of group A, and % denotes the bond to G¹;
Q-Z is %-CH₂—O—*, ′%-CH₂S(O)ₙ—*, or %-C(═O)—O—*, wherein % marks the bond of
Q to phenyl, and * the bond of Z to azetidin; and
R^{A4} is H or C(═O)R^{4A}, wherein
   R^{4A} is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₄-alkylcarbonyl, which aliphatic groups are unsubstituted or substituted with one or more radicals R⁴¹;
      C₃-C₆-cycloalkyl, C₃-C₆-halocycloalkyl which cyclic groups are unsubstituted or substituted with one or more R⁴²; C(═O)N(R⁴³)R⁴⁴, N(R⁴³)R⁴⁵, CH═NOR⁴⁶;
      phenyl, heterocycle, or hetaryl HET which rings are unsubstituted or partially or fully substituted with R^{A};
   R⁴¹ is independently OH, CN, C₁-C₆-alkoxy, C₁-C₆-haloalkoxy, S(O)ₙ—C₁-C₆-alkyl, S(O)ₙ—C₁-C₆-haloalkyl, C(═O)N(R⁴³)R⁴⁴,
      C₃-C₆-cycloalkyl, or C₃-C₆-halocycloalkyl which cycles are unsubstituted or substituted with one or more R⁴¹¹; or
      phenyl, heterocycle or hetaryl HET which rings are unsubstituted or partially or fully substituted with R^{A};
   R⁴¹¹ is independently OH, CN, C₁-C₂-alkyl, or C₁-C₂-haloalkyl;
   R⁴³ is H, or C—C₆-alkyl,
   R⁴⁴ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, or C₃-C₆-cycloalkyl, C₃-C₆-halocycloalkyl, C₃-C₆-cycloalkylmethyl, or C₃-C₆-halocycloalkylmethyl which rings are unsubstituted or substituted with a cyano;
   R⁴⁵ H, C₁-C₆-alkyl, C₁-C₆-haloalkyl, C₂-C₄-alkenyl, C₂-C₄-alkynyl, CH₂—CN, C₃-C₆-cycloalkyl, C₃-C₆-halocycloalkyl, C₃-C₆-cycloalkylmethyl, C₃-C₆-halocycloalkylmethyl, phenyl and hetaryl HET which aromatic rings are unsubstituted or partially or fully substituted with R^{A};
   R⁴² C₁-C₆-alkyl, C₁-C₆-haloalkyl, or a group as defined for R⁴¹;
   R⁴⁶ is independently H, C₁-C₆-alkyl, or C₁-C₆-haloalkyl;
   R^{A} is independently selected from halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, S(O)ₙ-C₁-C₄-alkyl, S(O)ₙ—C₁-C₄-haloalkyl, C₁-C₄-alkylcarbonyl, C₁-C₄-haloalkylcarbonyl, C(═O)N(R⁴³)R⁴⁴; or
   two R^{A} present on the same carbon atom of a saturated or partially saturated ring may form together ═O or ═S; or
   two R^{A} present on the same S or SO ring member of a heterocyclic ring may together form a group ═N(C₁-C₆-alkyl), ═NO(C₁-C₆-alkyl), ═NN(H)(C₁-C₆-alkyl) or ═NN(C₁-C₆-alkyl)₂;
A³ is CH₂—NR⁵C(═W)R⁶;
A⁴ is cyano;
R⁵ is independently selected from the meanings mentioned for R²;
R⁶ is H, CN, C₁-C₁₀-alkyl, C₃-C₈-cycloalkyl, C₂-C₁₀-alkenyl, C₂-C₁₀-alkynyl, which groups are unsubstituted, partially or fully halogenated and/or substituted with one or more same or different R⁸; or
   S(O)ₙR⁹, or C(═O)R⁸; or
   a 3- to 8-membered saturated, partially or fully unsaturated heterocyclic ring, which ring may contain 1, 2, 3, or 4 heteroatoms O, S, N, C═O and/or C═S as ring members, which heterocyclic ring is unsubstituted or partially or fully substituted with same or different halogen, CN, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, which groups are unsubstituted, or partially or fully substituted with same or different R⁸, or phenyl which may be partially or fully substituted with R¹¹;
or R⁵ and R⁶ together form a group ═C(R⁸)₂, ═S(O)ₘ(R⁹)₂, ═NR^{10a}, or ═NOR⁹;
R^{7a}, R^{7b} are each independently H, halogen, CN, C₁-C₆-alkyl, C₃-C₈-cycloalkyl, C₂-C₆-alkenyl, or C₂-C₆-alkynyl, which groups are unsubstituted, partially or fully halogenated and/or substituted with same or different R⁸;
each R⁸ is independently CN, N₃, NO₂, SCN, SF₅, C₃-C₈-cycloalkyl, C₃-C₈-halocycloalkyl, wherein the carbon chains may be substituted with one or more R¹³;
   Si(R¹²)₃, OR⁹, OSO₂R⁹, S(O)ₙR⁹, N(R^{10a})R^{10b}, C(═O)N(R^{10a})R^{10b}, C(═S)N(R^{10a})R^{10b}, C(═O)OR⁹, CH═NOR⁹,
   phenyl, which is unsubstituted or partially or fully substituted with same or different R¹⁶, or
   a 3-, 4-, 5-, 6- or 7-membered saturated, partially or fully unsaturated heterocyclic ring comprising 1, 2 or 3 heteroatoms N, O, and/or S as ring members, which ring is unsubstituted or partially or fully substituted with same or different R¹⁶, or
two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b}; or
two radicals R⁸, together with the carbon atoms of the alkyl, alkenyl, alkynyl or cycloalkyl group which they are bonded to, form a 3-, 4-, 5-, 6-, 7- or 8-membered saturated or partially unsaturated carbocyclic or heterocyclic ring, which heterocyclic ring comprises 1, 2, 3 or 4 heteroatoms N, O, and/or S as ring members, and which ring is unsubstituted, or partially or fully substituted with same or different R¹⁶; and
R⁸ as a substituent on a cycloalkyl ring may additionally be C₁-C₆-alkyl, C₁-C₆-haloalkyl, C₂-C₆-alkenyl, C₂-C₆-haloalkenyl, C₂-C₆-alkynyl, and C₂-C₆-haloalkynyl, which groups are unsubstituted, or partially or fully substituted with same or different R¹³; and
R⁸ in the groups C(═O)R⁸ and ═C(R⁸)₂ may additionally be H, halogen, C₁-C₆-alkyl, C₁-C₆-haloalkyl, C₂-C₆-alkenyl, C₂-C₆-haloalkenyl, C₂-C₆-alkynyl, or C₂-C₆-haloalkynyl, which groups are unsubstituted, or partially or fully substituted with same or different R¹³;
each R⁹ is independently H, CN, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, or C₂-C₆-haloalkynyl, which groups are unsubstituted, or partially or fully substituted with same or different R¹³, or
   C₁-C₆-alkyl-C(═O)OR¹⁵, C₁-C₆-alkyl-C(═O)N(R^{14a})R^{14b}, C₁-C₆-alkyl-C(═S)N(R^{14a})R^{14b}, C₁-C₆-alkyl-C(═NR¹⁴)N(R^{14a})R^{14b}, Si(R¹²)₃, S(O)ₙR¹⁵, S(O)ₙN(R^{14a})R^{14b}, N(R^{10a})R^{10b}, N═C(R¹³)₂, C(═O)R¹³, C(═O)N(R¹⁴)R^{14b}, C(═S)N(R^{14a})R^{14b}, C(═O)OR¹⁵, or
   phenyl, which is unsubstituted, or partially or fully substituted with R¹⁶; and
   a 3- to 7-membered saturated, partially or fully unsaturated heterocyclic ring comprising 1, 2 or 3 heteroatoms N, O, and/or S as ring members, which ring is unsubstituted, or partially or fully substituted with same or different R¹⁶; and
R⁹ in the groups S(O)ₙR⁹ and OSO₂R⁹ may additionally be C₁-C₆-alkoxy, or C₁-C₆-haloalkoxy;
R^{10a}, R^{10b} are independently from one another H, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, which groups are unsubstituted, or partially or fully substituted with same or different R¹³;
   C₁-C₆-alkyl-C(═O)OR¹⁵, C₁-C₆-alkyl-C(═O)N(R^{14a})R^{14b}, C₁-C₆-alkyl-C(═S)N(R^{14a})R^{14b}, C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio, S(O)ₙR¹⁵, S(O)ₙN(R^{14a})R^{14b}, C(═O)R¹³, C(═O)OR¹⁵, C(═O)N(R^{14a})R^{14b}, C(═S)R¹³, C(═S)SR¹⁵, C(═S)N(R^{14a})R^{14b}, C(═NR¹⁴)R¹³;
   phenyl, which is unsubstituted, or partially or fully substituted with same or different R¹⁶; and
   a 3-, 4-, 5-, 6- or 7-membered saturated, partially or fully unsaturated heterocyclic ring comprising 1, 2, 3 or 4 heteroatoms N, O, and/or S as ring members, which ring is unsubstituted, or partially or fully substituted with same or different R¹⁶, preferably unsubstituted or substituted HET; or
R^{10a} and R^{10b} together with the nitrogen atom they are bonded to form a 3- to 8-membered saturated, partially or fully unsaturated heterocyclic ring, which ring may additionally contain one or two heteroatoms N, O, and/or S as ring members, which ring is unsubstituted, or partially or fully substituted with same or different halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be partially or fully substituted with R¹⁶, and a 3-, 4-, 5-, 6,- or 7-membered saturated, partially or fully unsaturated heterocyclic ring comprising 1, 2 or 3 heteroatoms N, O, and/or S as ring members, which ring is unsubstituted, or partially or fully substituted with same or different R¹⁶; or
R^{10a} and R^{10b} together form a group ═C(R¹³)₂, ═S(O)ₘ(R¹⁵)₂, ═S(O)ₘR¹⁵N(R^{14a})R^{14b}, ═NR¹⁴, or ═NOR¹⁵;
R¹¹ is halogen, CN, N₃, NO₂, SCN, SF₅, C₁-C₁₀-alkyl, C₃-C₈-cycloalkyl, C₂-C₁₀-alkenyl, C₂-C₁₀-alkynyl, which groups are unsubstituted, partially or fully halogenated, and/or may be substituted with same or different R⁸, or
   OR⁹, NR^{10a}R^{10b}, S(O)ₙR⁹, Si(R¹²)₃;
   phenyl, which is unsubstituted, or partially or fully substituted with same or different R¹⁶; and
   a 3- to 7-membered saturated, partially or fully unsaturated aromatic heterocyclic ring comprising 1, 2, 3, or 4 heteroatoms N, O, and/or S as ring members, which ring is unsubstituted, or partially or fully substituted with same or different R¹⁶; or
two R¹¹ present on the same ring carbon atom of an unsaturated or partially unsaturated heterocyclic ring may together form a group ═O, ═C(R¹³)₂, ═S, ═S(O)ₘ(R¹⁵)₂, ═S(O)ₘR¹⁵N(R^{14a})R^{14b}, ═NR¹⁴, ═NOR¹⁵, or ═NN(R^{14a})R^{14b};
or two R¹¹ bound on adjacent ring atoms form together with the ring atoms to which they are bound a saturated 3- to 9-membered ring, which ring may contain 1 or 2 heteroatoms O, S, N, and/or NR¹⁴, and/or 1 or 2 groups C═O, C═S, C═NR¹⁴ as ring members, and which ring is unsubstituted, or partially or fully substituted with same or different halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be partially or fully substituted with same or different R¹⁶, and a 3- to 7-membered saturated, partially or fully unsaturated heterocyclic ring containing 1, 2, or 3 heteroatoms N, O, and/or S as ring members, which ring is unsubstituted, or partially or fully substituted with same or different R¹⁶;
each R¹² is independently C₁-C₄-alkyl and phenyl, which is unsubstituted, or partially or fully substituted with same or different C₁-C₄-alkyl;
each R¹³ is independently CN, NO₂, OH, SH, SCN, SF₅, C₁-C₆-alkoxy, C₁-C₆-haloalkoxy, SOₙ—C₁-C₆-alkyl, SOₙ—C₁-C₆-haloalkyl, Si(R¹²)₃, —C(═O)N(R^{14a})R^{14b}, C₃-C₈-cycloalkyl which is unsubstituted, partially or fully halogenated or substituted with 1 or 2 same or different C₁-C₄-alkyl, C₃-C₄-cycloalkyl, C₁-C₄-alkoxy, C₁-C₄-haloalkoxy and/or oxo; phenyl, benzyl, phenoxy, where the phenyl moiety may be substituted with one or more same or different R¹⁶; and a 3- to 7-membered saturated, partially or fully unsaturated heterocyclic ring containing 1, 2, or 3 heteroatoms N, O, and/or S, as ring members, which ring is unsubstituted, or partially or fully substituted with same or different R¹⁶; or
two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═N(C₁-C₆-alkyl) or ═NO(C₁-C₆-alkyl); and
R¹³ as a substituent of a cycloalkyl ring may additionally be C₁-C₆-alkyl, C₂-C₆-alkenyl or C₂-C₆-alkynyl, which groups are unsubstituted, partially or fully halogenated, or substituted with 1 or 2 CN, C₃-C₄-cycloalkyl, C₁-C₄-alkoxy, C₁-C₄-haloalkoxy, and oxo; and
R¹³ in groups ═C(R¹³)₂, N═C(R¹³)₂, C(═O)R¹³, C(═S)R¹³, and C(═NR¹⁴)R¹³ may additionally be H, halogen, C₁-C₆-alkyl, C₂-C₆-alkenyl, or C₂-C₆-alkynyl, which groups are unsubstituted, partially or fully halogenated, or substituted with 1 or 2 CN, C₃-C₄-cycloalkyl, C₁-C₄-alkoxy, C₁-C₄-haloalkoxy, and oxo;
each R¹⁴ is independently H, CN, C₁-C₆-alkoxy, C₁-C₆-haloalkoxy, SOₙ—C₁-C₆-alkyl, SOₙ—C₁-C₆-haloalkyl, Si(R¹²)₃;
   C₁-C₆-alkyl, C₂-C₆-alkenyl, C₂-C₆-alkynyl, which groups are unsubstituted, partially or fully halogenated, or substituted with 1 or 2 CN, C₁-C₄-alkoxy, C₁-C₄-haloalkoxy, SOₙ—C₁-C₄-alkyl, C₃-C₆-cycloalkyl which is unsubstituted or substituted with 1 or 2 substituents halogen and CN;
   and oxo;
   C₃-C₈-cycloalkyl which is unsubstituted, or partially or fully halogenated or substituted with 1 or 2 CN, C₁-C₄-alkyl, C₁-C₄-alkoxy, C₁-C₄-haloalkoxy, SOₙ—C₁-C₆-alkyl, C₃-C₄-cycloalkyl, C₃-C₄-cycloalkyl-C₁-C₄-alkyl-, which groups are unsubstituted, or substituted with 1 or 2 substituents selected from halogen and CN;
   phenyl, benzyl, pyridyl, phenoxy, which cyclic moieties are unsubstituted, or substituted with one or more same or different halogen, CN, NO₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₂-C₄-alkenyl, C₂-C₄-haloalkenyl, C₂-C₄-alkynyl, C₂-C₄-haloalkynyl, C₃-C₆-cycloalkyl, C₃—C₆-halocycloalkyl, and C₁-C₆-alkoxycarbonyl; and a 3-, 4-, 5- or 6-membered saturated, partially or fully unsaturated heterocyclic ring comprising 1, 2 or 3 heteroatoms N, O, and/or S as ring members, which ring is unsubstituted, or partially or fully substituted with same or different R¹⁶;
R^{14a} and R^{14b} independently of each other, have one of the meanings given for R¹⁴; or
R^{14a} and R^{14b}, together with the nitrogen atom to which they are bound, form a 3- to 7-membered saturated, partially, or fully unsaturated heterocyclic ring, wherein the ring may additionally contain 1 or 2 heteroatoms N, O, and/or S as ring members, which ring is unsubstituted, or partially or fully substituted with same or different halogen, C₁-C₄-alkyl, C₁-C₄-haloalkyl, C₁-C₄-alkoxy, or C₁-C₄-haloalkoxy; or
R^{14a} and R¹⁴ or R^{14b} and R¹⁴, together with the nitrogen atoms to which they are bound in the group C(═NR¹⁴)N(R^{14a})R^{14b}, form a 3- to 7-membered partially, or fully unsaturated heterocyclic ring, wherein the ring may additionally contain 1 or 2 heteroatoms N, O, and/or S as ring members, which ring is unsubstituted, or partially or fully substituted with same or different halogen, C₁-C₄-haloalkyl, C₁-C₄-alkoxy, or C₁-C₄-haloalkoxy;
each R¹⁵ is independently H, CN, Si(R¹²)₃
   C₁-C₆-alkyl, C₂-C₆-alkenyl, C₂-C₆-alkynyl, which groups are unsubstituted, partially or fully halogenated, or substituted with 1 or 2 radicals C₃-C₄-cycloalkyl, C₁-C₄-alkoxy, C₁-C₄-haloalkoxy, SOₙ—C₁-C₆-alkyl, or oxo;
   C₃-C₈-cycloalkyl which is unsubstituted, partially or fully halogenated or substituted with 1 or 2 radicals C₁-C₄-alkyl, C₃-C₄-cycloalkyl, C₁-C₄-alkoxy, C₁-C₄-haloalkoxy, SOₙ—C₁-C₆-alkyl, or oxo;
   phenyl, benzyl, pyridyl, and phenoxy, which rings are unsubstituted, partially or fully halogenated, or substituted with 1, 2 or 3 substituents C₁-C₆-alkyl, C₁-C₆-haloalkyl, C₁-C₆-alkoxy, C₁-C₆-haloalkoxy, or (C₁-C₆-alkoxy)carbonyl;
each R¹⁶ is independently halogen, NO₂, CN, OH, SH, C₁-C₆-alkoxy, C₁-C₆-haloalkoxy, SOₙ—C₁-C₆-alkyl, SOₙ—C₁-C₆-haloalkyl, C₁-C₄-alkylcarbonyl, C₁-C₄-haloalkylcarbonyl, C₁-C₄-alkoxycarbonyl, C₁-C₄-haloalkoxycarbonyl, aminocarbonyl, C₁-C₄-alkylaminocarbonyl, di-(C₁-C₄-alkyl)-aminocarbonyl, Si(R¹²)₃;
   C₁-C₆-alkyl, C₂-C₆-alkenyl, C₂-C₆-alkynyl, which groups are unsubstituted, partially or fully halogenated, or substituted with 1 or 2 radicals CN, C₃-C₄-cycloalkyl, C₁-C₄-alkoxy, C₁-C₄-haloalkoxy, or oxo;
   C₃-C₈-cycloalkyl which is unsubstituted, partially or fully halogenated or substituted with 1 or 2 radicals CN, C₁-C₄-alkyl, C₃-C₄-cycloalkyl, C₁-C₄-alkoxy, C₁-C₄-haloalkoxy, or oxo;
   phenyl, benzyl, pyridyl and phenoxy, which rings are unsubstituted, partially or fully halogenated, or substituted with 1, 2 or 3 substituents C₁-C₆-alkyl, C₁-C₆-haloalkyl, C₁-C₆-alkoxy, C₁-C₆-haloalkoxy, or (C₁-C₆-alkoxy)carbonyl; or
two R¹⁶ present together on the same atom of an unsaturated or partially unsaturated ring may be ═O, ═S, ═N(C₁-C₆-alkyl), ═NO—C₁-C₆-alkyl, ═CH(C₁-C₄-alkyl), or ═C(C₁-C₄-alkyl)₂; or
two R¹⁶ on two adjacent carbon atoms form together with the carbon atoms they are bonded to a 4- to 8-membered saturated, partially or fully unsaturated ring, wherein the ring may contain 1 or 2 heteroatoms N, O, and/or S as ring members, which ring is unsubstituted, or partially or fully substituted with same or different halogen, C₁-C₄-alkyl, C₁-C₄-haloalkyl, C₁-C₄-alkoxy, or C₁-C₄-haloalkoxy;
each n is independently 0, 1, or 2; and
each m is independently 0, or 1;

wherein the shown enantiomer has at least 50% ee;

by condensation of a ketone of formula II with an acetyl compound of formula III,

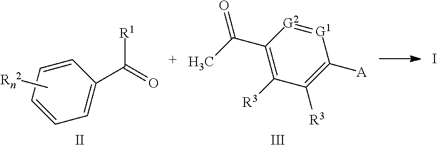

wherein the variables have the meanings given for formula I, in the presence of a catalyst of formula IV

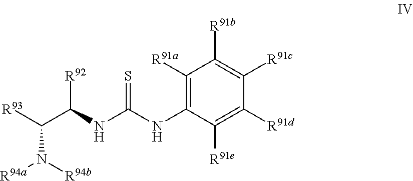

wherein the variables have following meanings:

in case IVa (Takemoto's Catalyst):
R^{91a} to R^{91e} are independently from one another selected from H, CN, NO₂, and C₁-C₆-alkoxycarbonyl;
R⁹² and R⁹³ together with the carbon atoms they are bound to form a cyclohexyl ring;
R^{94a}, R^{94b} are selected from C₁-C₃-alkyl;

in case IVb:
R^{91a} to R^{91e} are independently from one another selected from H, CN, NO₂, and C₁-C₆-alkoxycarbonyl;
R⁹² is selected from 6-methoxy-4-quinolyl, and 4-quinolyl;

R⁹³, R^{94a} and R^{94b} together with the bridging nitrogen atom form a bridged ring system containing 5 to 10 ring members which is unsubstituted or substituted with one or more halogen, C₁-C₄-alkyl, C₁-C₂-haloalkyl, or C₂-C₄-alkenyl, wherein two substitutents bound to the same C-atom may form a ═CH₂ group;

preferably the formula IV compound is selected from IVb-1, IVb-2, IVb-3, and IVb-4.

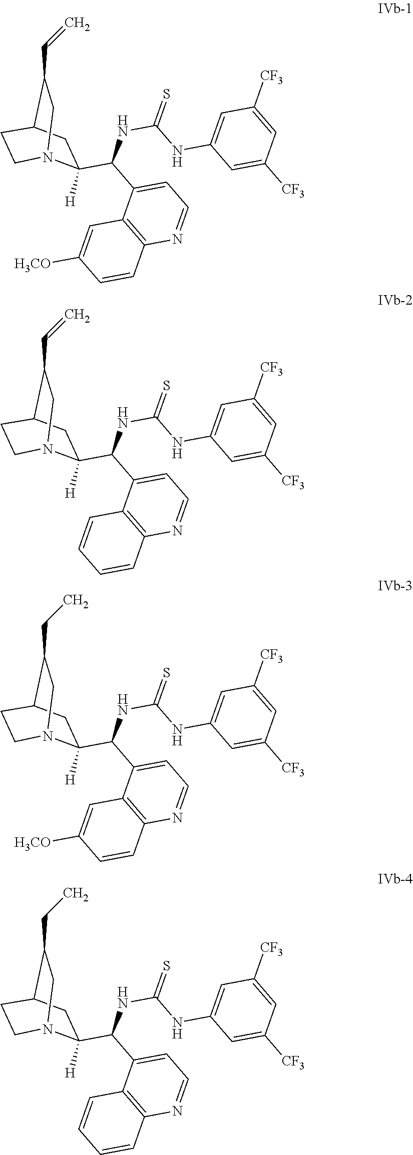

Compounds of formula I are valuable intermediates for the preparation of active compounds of formula V,

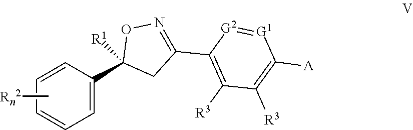

wherein the ee is at least 95%, and the variables are as defined in general and preferred embodiments for formula I, and A is A¹ or A².

These isoxazoline active compounds V and their pesticidal activity are generally known from WO 2005/085216, WO 2007/026965, WO 2009/00289, WO 2011/067272, WO 2012/120399, WO 2014/090918, WO 2016/102482, and PCT/EP2018/060439.

WO2017/176948 describes a process for preparing compounds of formula V involving an irreversible and not stereospecific Michael addition yielding both isomers. The undesired isomer cannot be recycled.

The present invention, however, forms an optically enriched aldol intermediate if formula I which through a retro aldol reaction offers the possibility of recycling the undesired isomer—if formed—back to the starting materials II and III.

However, optically enriched compounds of formula V cannot be prepared in good yield by the processes disclosed in the art. Objective task for the present invention therefore is providing an economical, industrially applicable manufacturing process for optically enriched compounds of formula V, and to the active compounds of formula IV. This task is achieved by the process defined in the outset. The presence of a catalyst as defined herein in the reaction of compounds II and III ensures a quick and complete transformation at moderate temperatures.

The condensation of a ketone of formula II with an acetyl compound of formula III, wherein the variables have the meanings given in the outset, is usually carried out at temperatures of from 0° C. to +150° C., preferably from +10° C. to +80° C., in an inert solvent, in the presence of catalyst of formula IV [cf. P. Wang et al, Organic Letters, 19(10), 2634-2637; 2017]

Suitable solvents are aliphatic hydrocarbons such as pentane, hexane, cyclohexane, and petrol ether, aromatic hydrocarbons such as toluene, o-, m-, and p-xylene, halogenated hydrocarbons such as CH₂Cl₂, CHCl₃, dichloroethane and chlorobenzene, ethers such as diethyl ether, diisopropylether, tert.-butylmethyl ether (TBME), 1,4-dioxane, anisole, and tetrahydrofurane (THF), nitrils such as acetonitrile, and propionitrile, alcohols such as methanol, ethanol, n-propanol, isopropanol, n-butanol, and tert.-butanol, preferably aliphatic hydrocarbons or halogenated hydrocarbons such as dichloroethane. It is also possible to use mixtures of the solvents mentioned.

Starting materials of formulae II and III, resp. required for preparing the compounds I are commercially available or known from the literature (cf. WO 2012/120399, WO 2015/128358, WO 2016/102490) or can be prepared as outlined above, or in accordance with the literature cited.

The catalyst is used in 0.01 to 0.5, preferably 0.01 to 0.2, particularly about 0.02 to 0.1 mol equivalents of compound II. The starting materials are generally reacted with one another in equimolar amounts. In terms of yield, it may be advantageous to employ an excess of II, based on III.

Furthermore, the invention also relates to a process for the manufacture of compounds of formula VI starting from formula I compounds which are reacted with hydroxylamine VII to yield the Z-oximes VI

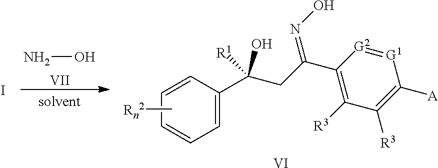

wherein the oxime group is predominately, such as at least by 90%, in the shown Z-formation, and the variables are as defined in general and preferred embodiments for formula I.

This transformation is usually carried out at temperatures of from 0° C. to +100° C., preferably from +10° C. to +50° C., in an inert solvent, in the presence of an acid or a base [cf. E. Lodge et al, Journal of the American Chemical Society, 109(11), 3353-61; 1987].

Suitable solvents are unsubstituted or substituted pyridines, if appropriate in combination with aliphatic hydrocarbons such as pentane, hexane, cyclohexane, and petrol ether, aromatic hydrocarbons such as toluene, o-, m-, and p-xylene, halogenated hydrocarbons such as methylene chloride, chloroform, and chlorobenzene, ethers such as diethylether, diisopropylether, TBME, dioxane, anisole, and THF, nitrils such as acetonitrile, and propionitrile, alcohols such as methanol, ethanol, n-propanol, isopropanol, n-butanol, and tert.-butanol, preferably pyridine, 2,6-lutidine, 2,3-lutidine, 2,5-lutidine, 2-methyl pyridine, neat or as mixture with one another. It is also possible to use mixtures of the solvents mentioned. The solvent system preferably consists predominantly of pyridine, 2,6-lutidine, 2,3-lutidine, 2,5-lutidine, 2-methyl pyridine, neat or as mixture with one another, and can contain up to 50% by weight of other solvents.

Suitable acids and acidic catalysts are in general inorganic acids such as hydrofluoric acid, hydrochloric acid, hydrobromic acid, sulphuric acid und perchloric acid, Lewis acids, such as BF₃, AlCl₃, FeCl₃, SnCl₄, TiCl₄ and ZnCl₂, moreover organic acids such as formic acid, acetic acid, propionic acid, oxalic acid, toluene sulphonic acid, benzene sulphonic acid, camphor sulphonic acid, citric acid, and trifluoro acetic acid. The acids are generally employed in catalytic amounts; however, they can also be used in equimolar amounts, in excess or, if appropriate, as solvent.

For practical reasons hydroxylamine VII is used in the form of an acid addition salt, preferably as halogenide or sulfate, preferably halogenide, particularly as HCl addition salt.

Suitable bases include pyridine, 2,6-lutidine, 2,3-lutidine, 2,5-lutidine, 2-methyl pyridine, neat or as mixture with one another, NaOH, KOH, sodium acetate, potassium acetate, NaHCO₃, Na₂CO₃, KHCO₃, K₂CO₃. The base is generally employed in excess based on VII.

The starting materials are generally reacted with one another in equimolar amounts. In terms of yield, it may be advantageous to employ an excess of VII, based on 1.

Compounds of formula VI, wherein the variables are as defined and preferred for formula I, are novel.

Furthermore, the invention also relates to a process for the manufacture of compounds of formula V by cyclisation of formula VI compounds to yield compounds of formula Va. In formula Va the variables are as defined for formula VI. If group A in formula VI and Va is C(═W)N(R⁵)R⁶, or A², formula Va may correspond to the active compound of formula V.

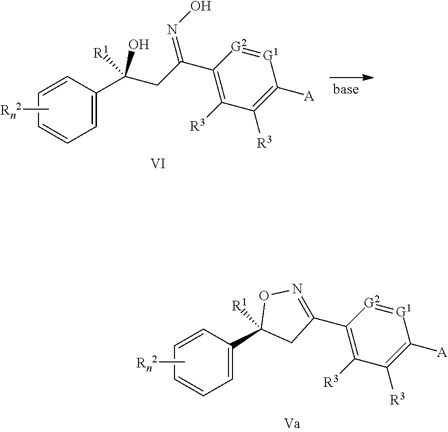

This transformation is usually carried out at temperatures of from −50° C. to +50° C., preferably from −5° C. to +25° C., in an inert solvent, in the presence of a base and an activating agent [cf. J. Chem. Soc. Chem. Commun. 1983, 873-875; US2010/179194; Org. Lett. 2017, 19, 2634-2637].

Suitable solvents are aliphatic hydrocarbons such as pentane, hexane, cyclohexane, and petrol ether, aromatic hydrocarbons such as toluene, o-, m-, and p-xylene, halogenated hydrocarbons such as methylene chloride, chloroform, and chlorobenzene, ethers such as diethylether, Diisopropylether, TBME, dioxane, anisole, and THF, nitrils such as acetonitrile, and propionitrile, moreover dimethyl formamide (DMF), and dimethylacetamide (DMA), preferably aromatic hydrocarbons and ethers (toluol, THF). It is also possible to use mixtures of the solvents mentioned.

Suitable bases are, in general, inorganic compounds, such as-alkali metal and alkaline earth metal hydrides, such as LiH, NaH, KH and CaH₂, moreover organic bases, e.g. tertiary amines, such as trimethylamine, triethylamine (NEt₃), triisopropylethylamine, and N-methylpiperidine, pyridine, substituted pyridines, such as collidine, lutidine and 4-dimethylaminopyridine, and also bicyclic amines; moreover alkali metal amides, e.g. alkali metal diisopropylamides, such as lithium diisopropylamide; other alkali metal amides such as lithium bis(trimethylsilyl)amide (LiHMDS), or hexamethyldisilazanes, such as lithium hexamethyldisilazane, sodium hexamethyldisilazane, potassium hexamethyldisilazane, or lithium tetramethylpiperidide. Particular preference is given to lithium dissopropylamide, lithium hexamethyldisilazane, sodium hexamethyldisilazane, potassium hexamethyldisilazane. The bases are generally employed in catalytic amounts; however, they can also be used in equimolar amounts, in excess or, if appropriate, as solvent.

Suitable activating agents are halogenating agents, which are usually selected from chlorinating agents and brominating agents, such as oxalylchloride, SOCl₂, PBr₃, and PBr₅, PCl₃, and PCl₅, sulfonic acid chlorides, such as toluoisulfonic acid chloride (TsCl) and methylsulfonic acid chloride (MsCl), preferably from SOCl₂ and oxalylchloride.

The starting materials are generally reacted with one another in equimolar amounts. In terms of yield, it may be advantageous to employ an excess of the activating agent, based on VI.

If in compounds Va group A is A¹ or A³ different from group A in the envisaged final active compounds V, the process also comprises the amidation of Va with an appropriate amine VIII under conditions known in the art, e.g. WO2004/22536.

Compounds of formula V can be prepared by reacting carboxylic acids or acid derivatives of formula Va′ with an amine of formula VIII in an amidation reaction.

In formula Va′ the variables are as defined for formula V, and
Y is OR⁹, wherein R⁹ is H or a leaving group, preferably C₁-C₆-alkoxy, such as OCH₃ or OC₂H₅, or
Y is N(R⁵)R⁶, wherein R⁵ and R⁶ are preferably H or C₁-C₆-alkyl.

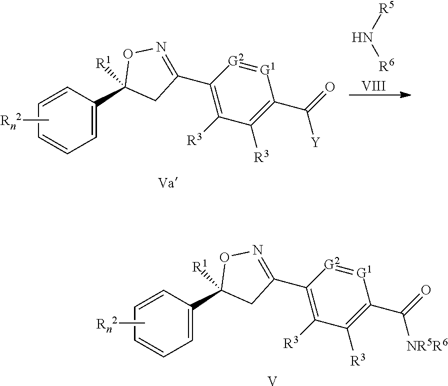

The amidation reaction is preferably carried out by direct reaction with the amine VIII, or by prior transformation of carboxylic acids of formula Va′ (Y is OH) with oxalyl chloride [(COCl)₂] or thionylchloride (SOCl₂) to the corresponding acid chlorides of formula Vb, followed by reaction with an amine of formula VIII. The reaction is preferably carried out in the presence of an organic base such as, NEt₃, N-ethyl-N,N-diisopropylamine, pyridine, or substituted pyridines such as collidine or lutidine. Optionally a nucleophilic catalyst such as 4-(N,N-dimethylamino)pyridine (“DMAP”) can be employed in the reaction. Suitable solvents are halogenated hydrocarbons such as, dichloromethane, chloroform, and chlorobenzene, or polar aprotic solvents such as THF, 1,4-dioxane, and N,N-dimethylformamide (DMF), or aromatic hydrocarbons such as benzene, toluene, o-, m-, and p-xylene, or mixtures thereof. The transformation is usually carried out at temperatures from −40° C. to 100° C., preferably from 0° C. to 30° C. The starting materials are generally reacted with one another in equimolar amounts. In terms of yield, it may be advantageous to employ an excess of VIII, based on Va.

Compounds of formula V with A being A³ can preferably be prepared by reduction of nitrils of formula Va wherein A is A⁴ (formula Va″) to the corresponding amine of formula Vc, and subsequent acylation of Vc with a carboxylic acid derivative of formula IX. In formula Va″ the variables are as defined for formula V.

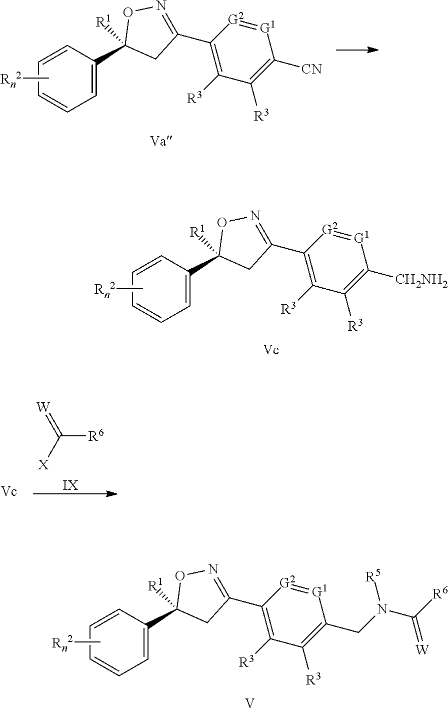

The reduction of Va″ to Vc is usually carried out at temperatures of from −10° C. to +110° C., preferably from 0° C. to +60° C., in an inert solvent, in the presence of a base, a reducing agent and a catalyst [cf. JP 2010235590].

Suitable solvents are aliphatic hydrocarbons such as pentane, hexane, cyclohexane, and petrol ether, aromatic hydrocarbons such as toluene, o-, m-, and p-xylene, halogenated hydrocarbons such as methylene chloride, chloroform, and chlorobenzene, ethers such as diethylether, diisopropylether, TBME, dioxane, anisole, and THF, nitrils such as acetonitrile, and propionitrile, alcohols such as methanol, ethanol, n-propanol, isopropanol, n-butanol, and tert.-butanol, moreover water; preferably alcohols, ethers and water. It is also possible to use mixtures of the solvents mentioned.

Suitable bases are, in general, inorganic compounds, such as alkali metal and alkaline earth metal hydroxides, such as LiOH, NaOH, KOH and Ca(OH)₂, alkali metal and alkaline earth metal oxides, such as Li₂O, Na₂O, CaO, and MgO, alkali metal and alkaline earth metal hydrides, such as LiH, NaH, KH and CaH₂, alkali metal and alkaline earth metal carbonates, such as Li₂CO₃, K₂CO₃ and CaCO₃, and also alkali metal bicarbonates, such as NaHCO₃, moreover organic bases, e.g. tertiary amines, such as trimethylamine, NEt₃, diisopropylethylamine and N-methylpiperidine, pyridine, substituted pyridines, such as collidine, lutidine and 4-dimethylaminopyridine, and also bicyclic amines. Particular preference is given to alkali metal and alkaline earth metal carbonates and alkali metal bicarbonates, such as NaHCO₃.

The bases are generally employed in catalytic amounts; however, they can also be used in equimolar amounts or in excess.

Suitable catalysts are nickel carbonyl, Raney nickel or nickel dichloride.

Suitable reducing agents are hydrogen gas or alkali metal hydrides such as sodium borohydride or lithium borohydride.

The starting materials are generally reacted with one another in equimolar amounts. In terms of yield, it may be advantageous to employ an excess of II, based on III.

The acylation is usually carried out at temperatures of from −10° C. to 110° C., preferably from 0° C. to 60° C., in an inert solvent, in the presence of a base and a catalyst [cf. Organic Letters, 18(23), 5998-6001; 2016].

Suitable solvents are aliphatic hydrocarbons such as pentane, hexane, cyclohexane, and petrol ether, aromatic hydrocarbons such as toluene, o-, m-, and p-xylene, halogenated hydrocarbons such as methylene chloride, chloroform, and chlorobenzene, ethers such as diethylether, diisopropylether, TBME, dioxane, anisole, and THF, nitrils such as acetonitrile, and propionitrile, alcohols such as methanol, ethanol, n-propanol, isopropanol, n-butanol, and tert.-butanol, moreover water; preferably halogenated hydrocarbons and aromatic hydrocarbons. It is also possible to use mixtures of the solvents mentioned.

Suitable bases are, in general, inorganic compounds, such as alkali metal and alkaline earth metal hydroxides, such as LIOH, NaOH, KOH and Ca(OH)₂, alkali metal and alkaline earth metal oxides, such as Li₂O, Na₂O, CaO, and MgO, alkali metal and alkaline earth metal hydrides, such as LiH, NaH, KH and CaH₂, alkali metal and alkaline earth metal carbonates, such as Li₂CO₃, K₂CO₃ and CaCO₃, and also alkali metal bicarbonates, such as NaHCO₃, moreover organic bases, for example tertiary amines, such as trimethylamine, NEt₃, diisopropylethylamine and N-methylpiperidine, pyridine, substituted pyridines, such as collidine, lutidine and 4-dimethylaminopyridine, and also bicyclic amines. Particular preference is given to alkali metal and alkaline earth metal carbonates and alkali metal bicarbonates, such as NaHCO₃.

The bases are generally employed in catalytic amounts; however, they can also be used in equimolar amounts, in excess or, if appropriate, as solvent.

Suitable catalysts are for example 4-N,N-dimethyl aminopyridine.

The starting materials are generally reacted with one another in equimolar amounts. In terms of yield, it may be advantageous to employ an excess of II, based on III.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

However, if the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

Furthermore, in one embodiment the invention relates to a process for the manufacture of compounds of formula V comprising the steps of reacting formulae I and III to the chiral aldols I, further reacting I to the Z-oximes VI, and cyclisation and amidation VI to the final active compounds V.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine, or iodine, in particular fluorine, chlorine, or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms.

The term “alkylsulfonyl” (S(═O)₂-alkyl) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “alkylcarbonyl” refers to an alkyl group as defined above, which is bonded via the carbon atom of a carbonyl group (C═O) to the remainder of the molecule.

The term “alkoxycarbonyl” refers to an alkylcarbonyl group as defined above, which is bonded via an oxygen atom to the remainder of the molecule.

The term “alkenyl” as used herein denotes in each case a singly unsaturated hydrocarbon radical having usually 2 to 10, frequently 2 to 6, preferably 2 to 4 carbon atoms.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radical having usually 2 to 10, frequently 2 to 6, preferably 2 to 4 carbon atoms.

The term “cycloalkyl” as used herein and in the cycloalkyl moieties of cycloalkoxy and cycloalkylthio denotes in each case a monocyclic cycloaliphatic radical having usually from 3 to 10 or from 3 to 6 carbon atoms, such as cyclopropyl, cyclobutyl, cyclopentyl, cyclohexyl, cycloheptyl, cyclooctyl, cyclononyl, and cyclodecyl, or cyclopropyl (c-C₃H₅), cyclobutyl (c-C₄H₇), cyclopentyl (c-C₅H₉), and cyclohexyl (c-C₆H₁₁).

The term “halocycloalkyl” as used herein and in the halocycloalkyl moieties of halocycloalkoxy and halocycloalkylthio denotes in each case a monocyclic cycloaliphatic radical having usually from 3 to 10 C atoms or 3 to 6 C atoms, wherein at least one, e.g. 1, 2, 3, 4 or 5 of the hydrogen atoms, are replaced by halogen, in particular by fluorine or chlorine.

The term “carbocycle” or “carbocyclyl” includes in general a 3- to 12-membered, preferably a 3- to 8-membered or a 5- to 8-membered, more preferably a 5- or 6-membered monocyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 5- or 6-membered, in particular 6-membered monocyclic heterocyclic non-aromatic radicals. The heterocyclic non-aromatic radicals usually comprise 1, 2 or 3 heteroatoms selected from N, O and S as ring members, wherein S-atoms as ring members may be present as S, SO or SO₂.

The term “hetaryl” includes monocyclic 5- or 6-membered heteroaromatic radicals comprising as ring members 1, 2, or 3 heteroatoms selected from N, O and S.

The terms “heterocyclyolalkyl” and “hetarylalkyl” refer to heterocyclyl or hetaryl, resp., as defined above which are bound via a C₁-C₄-alkyl group, in particular a methyl group (=heterocyclylmethyl or hetarylmethyl, resp.), to the remainder of the molecule.

With respect to the variables, the particularly preferred embodiments of the intermediates correspond to those of the compounds of the formula I.

In a particular embodiment, the variables of the compounds of the formula I have the following meanings, these meanings, both on their own and in combination with one another, being particular embodiments of the compounds of formula I.

In the compounds of the inventive process R¹ is preferably fluoromethyl, in particular CF₃.

The phenyl ring in formulae I, II, V, and VI bearing the R²ₙ substitution is preferably a group P

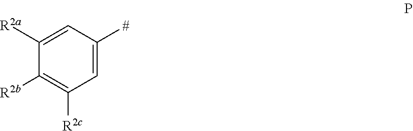

R^{2a} is preferably selected from F, Cl, Br, CF₃, and OCF₃.

R^{2b} and R^{2c} are independently preferably selected from H, F, Cl, Br, CF₃, and OCF₃.

Particularly preferred is each one of the following combinations of R^{2a}, R^{2b}, and R^{2c} wherein each line of Table A denotes a substitution pattern of the phenyl ring P bearing the R^{2a}, R^{2b}, and R^{2c} moieties.

TABLE A

| No. | R^{2a} | R^{2b} | R^{2c} |
|---|---|---|---|
| A-1 | F | F | H |
| A-2 | F | H | F |
| A-3 | F | F | F |
| A-4 | F | Cl | F |
| A-5 | F | Br | F |
| A-6 | F | H | Cl |
| A-7 | F | H | Br |
| A-8 | Cl | F | H |
| A-9 | Cl | H | Cl |
| A-10 | Cl | Cl | Cl |
| A-11 | Cl | F | Cl |
| A-12 | Cl | Br | Cl |
| A-13 | Cl | H | Br |
| A-14 | Br | F | H |
| A-15 | Br | H | Br |
| A-16 | Br | F | Br |
| A-17 | Br | Cl | Br |
| A-18 | CF₃ | H | H |
| A-19 | CF₃ | H | F |
| A-20 | CF₃ | H | Cl |
| A-21 | CF₃ | H | Br |
| A-22 | CF₃ | H | CF₃ |
| A-23 | CF₃ | F | F |
| A-24 | CF₃ | F | Cl |
| A-25 | CF₃ | Cl | Cl |
| A-26 | CF₃ | F | H |
| A-27 | OCF₃ | H | F |
| A-28 | OCF₃ | H | Cl |
| A-29 | OCF₃ | F | H |
| A-30 | OCF₃ | H | CF₃ |
| A-31 | OCF₃ | H | H |

Groups A-8, A-9, and A-11 are more preferred patterns in formula I, III, V, Va, and VI compounds. A-11 is particularly preferred.

R³ is preferably H, halogen, or CH₃.

In a preferred embodiment G¹ and G² represent each CR³, particularly G¹ is CH and G² is C—Cl, or C—CH₃.

In another embodiment G¹ and G² represent each CR³, wherein the two R³ form a five- or six-membered saturated carbocyclic ring, or a dihydrofurane.

In another embodiment Gi and G² together form a sulfur atom.

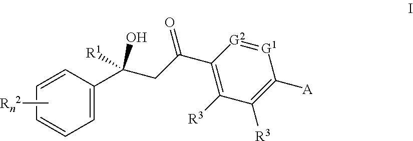

A preferred embodiment relates to the process for obtaining compounds V wherein A is A¹.

As catalyst IV preferably compound IVa-1 (1-[3,5-Bis(trifluoromethyl)phenyl]-3-[(1R,2R)-(−)-2-(dimethylamino)cyclohexyl]thiourea (“R,R-TUC”)) is used. This compound is known from U.S. Pat. No. 7,632,970.

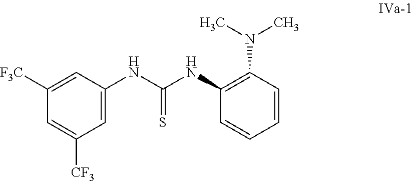

In another embodiment, compound IVb-1 (cf. Vakulya et al, Organic Letters (2005), 7(10), 1967-1969)) is used as catalyst

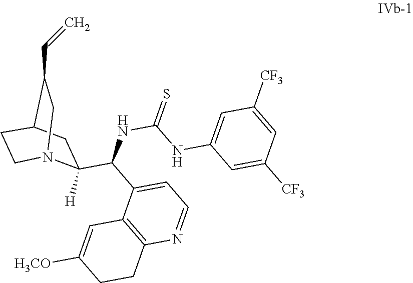 [SMILES: C=C[C@H]1CN2CCC1C[C@H]2[C@@H](NC(=S)Nc1cc(C(F)(F)F)cc(C(F)(F)F)c1)c1ccnc2c1C=C(OC)CC2]

In another embodiment, the catalyst is selected from compounds IVa-2, IVa-3, and IVa-4, which are known in the art (cf. Wang et al, Chemistry—A European Journal (2009), 15(3), 589-592; McCooey et al, Angewandte Chemie, International Edition (2005), 44(39), 6367-6370; Tan et al, Chemistry—A European Journal (2012), 18(21), 6414).

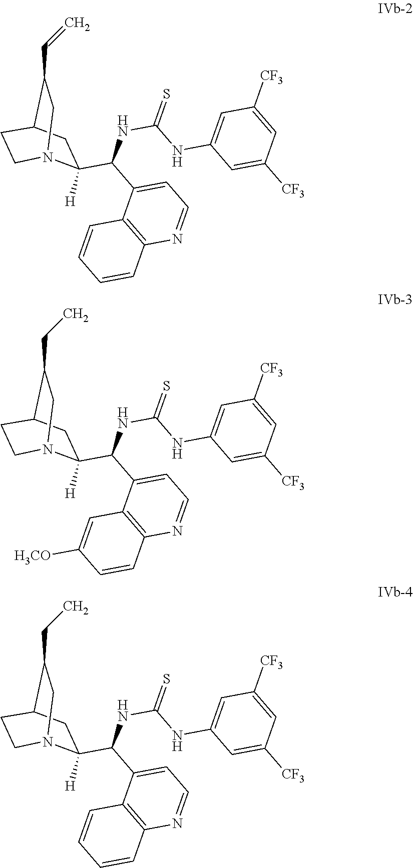

The catalyst IV is used preferably in an amount of 0.1-100 mol %, more preferred in 0.5-50 mol %, particularly in 1-20 mol % relative to formula II or III compounds.

The processes for obtaining compounds V wherein A is A¹ start preferably from compounds of formula III wherein A is C(═O)Y, and Y is OR⁹, preferably OH, or C₁-C₄-alkoxy, or NR⁵R⁶, wherein R⁵ and R⁶ are H or C₁-C₄-alkyl, preferably Y is NHCH₃. Particularly preferred A group in compounds I, III, Va, and VI is an C₁-C₄-alkylester, such as C(═O)OCH₃.

In A¹ the variables R⁵ and R⁶ have preferably following meanings:
R⁵ is preferably H, C₁-C₄-alkyl;
R⁶ is preferably H, C₁-C₆-alkyl, C₂-C₆-alkenyl, which groups are substituted with one or more same or different R⁸, wherein
R⁸ is preferably C₃-C₈-cycloalkyl, C₃-C₈-halocycloalkyl, wherein the carbon chains may be substituted with one or more R¹³;
   S(O)ₙR⁹, N(R^{10a})R^{10b}, C(═O)N(R^{10a})R^{10b}, C(═S)N(R^{10a})R^{10b}, C(═O)OR⁹, CH═NOR⁹,
   phenyl, which is unsubstituted or partially or fully substituted with same or different R¹⁶, or a 3-, 4-, 5-, 6- or 7-membered saturated, partially or fully unsaturated heterocyclic ring comprising 1, 2 or 3 heteroatoms N, O, and/or S as ring members, which ring is unsubstituted or partially or fully substituted with same or different R¹⁶, or

a 5-membered saturated heteromonocyclic ring containing 1, or 2 heteroatoms N, O, and/or S as ring members, which ring is unsubstituted, or substituted with one or more same or different R¹¹, preferably the unsubstituted or substituted HET;

two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
R⁹ is preferably H, CN, C₁-C₆-alkyl, C₁-C₆-haloalkyl;
R¹¹ C₁-C₁₀-alkyl, which is unsubstituted, partially or fully halogenated, and/or may be substituted with same or different R⁸, or
   OR⁹, NR^{10a}R^{10b}, S(O)ₙR⁹;
   two R¹¹ present on the same ring carbon atom of an unsaturated or partially unsaturated heterocyclic ring may together form a group ═O, ═C(R¹³)₂, ═S, ═S(O)ₘ(R¹⁵)₂, ═S(O)ₘR¹⁵N(R^{14a})R^{14b}, ═NR¹⁴, ═NOR¹⁵, or ═NN(R^{14a})R^{14b}.

Another embodiment relates to the process for obtaining compounds V wherein A is A², preferably wherein Q-Z is %-CH₂—O—*, and R⁴ is C₁-C₄-alkylcarbonyl wherein the terminal C-atom of the alkyl is substituted with S(O)ₙ-C₁-C₄-alkyl.

Another embodiment relates to the process for obtaining compounds V wherein A is A³, preferably CH₂—NR⁵C(═O)R⁶, wherein R⁵ is H or CH₃, and R⁶ is H, C₁-C₆-alkyl, C₂-C₆-alkenyl, which groups are substituted with one or more same or different R⁸, wherein R⁸ is as defined and preferred above.

Compounds I, Va, and VI wherein A is A⁴ are preferred intermediates in the inventive process.

The process is particularly suitable for synthesis of following active compounds of formula V, which correspond to formulae V.A, and V.B, wherein the variables are as defined and preferred above:

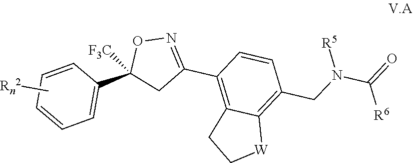

wherein W is CH or O; and

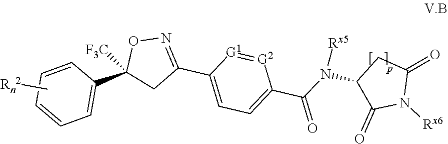

wherein p is 1 or 2; R^{x5} is H or CH₃, and R^{x6} is C₁-C₆-alkyl, C₁-C₄-haloalkyl, C₃-C₆-alkenyl, C₃-C₆-alkynyl, which groups may be substituted with C(═O)OR^{a1}, C(═O)N(R^{a2})R^{a3}, CH═NOR^{a1}, and phenyl, benzyl, which rings are unsubstituted or substituted with halogen, C₁-C₄-alkyl, or C₁-C₄-haloalkyl; wherein R^{a1} is C₁-C₆-alkyl, R^{a2} and R^{a3} are each H or C₁-C₆-alkyl, C₁-C₆-haloalkyl, C₂-C₄-alkenyl, C₂-C₄-alkynyl;

preferably R^{x6} is CH₃, C₂H₅, CH₂(CH₃)₂, CH₂CH═CH₂, CH₂CF₃, CH₂CH₂CF₃, CH₂C₆H₅, CH₂C(═O)OCH₃,

The process is furthermore particularly suitable for synthesis of following active compounds V.1, V.2, V.3, V.4, V.5, and V.6 of formula V which are known in the art (cf.: WO 2011067272; WO 2005085216; WO 200900289; WO 2014090918; WO 2007026965; WO 2012120399):

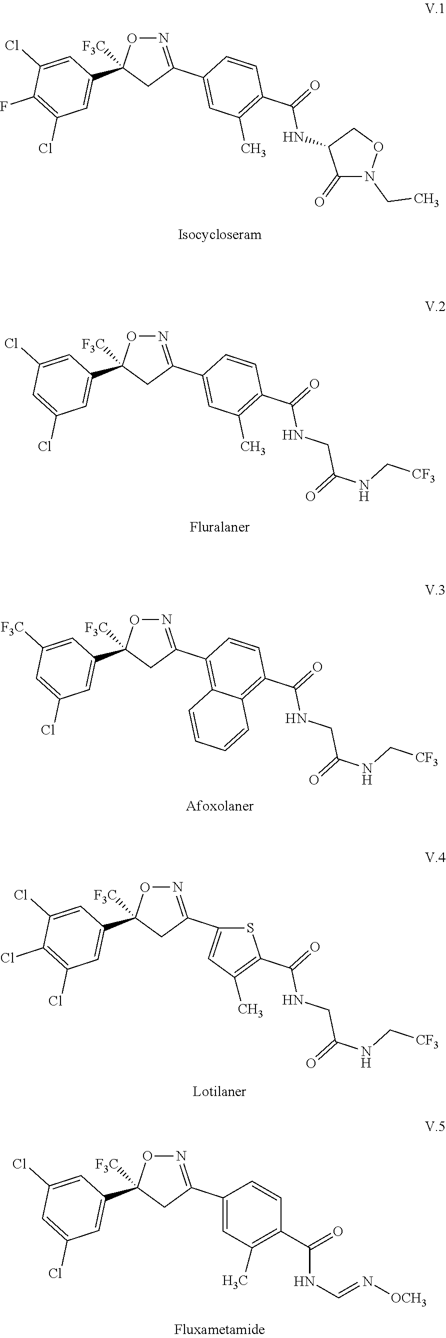 [SMILES: CCN1OC[C@@H](NC(=O)c2ccc(C3=NO[C@@](c4cc(Cl)c(F)c(Cl)c4)(C(F)(F)F)C3)cc2C)C1=O ; Cc1cc(C2=NO[C@@](c3cc(Cl)cc(Cl)c3)(C(F)(F)F)C2)ccc1C(=O)NCC(=O)NCC(F)(F)F ; O=C(CNC(=O)c1ccc(C2=NO[C@@](c3cc(Cl)cc(C(F)(F)F)c3)(C(F)(F)F)C2)c2ccccc12)NCC(F)(F)F ; Cc1cc(C2=NO[C@@](c3cc(Cl)c(Cl)c(Cl)c3)(C(F)(F)F)C2)sc1C(=O)NCC(=O)NCC(F)(F)F ; CO/N=C/NC(=O)c1ccc(C2=NO[C@@](c3cc(Cl)cc(Cl)c3)(C(F)(F)F)C2)cc1C]

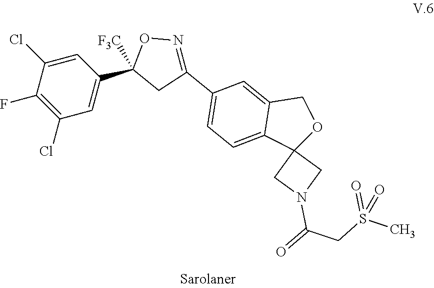 [SMILES: CS(=O)(=O)CC(=O)N1CC2(C1)OCc1cc(C3=NO[C@@](c4cc(Cl)c(F)c(Cl)c4)(C(F)(F)F)C3)ccc12]

Accordingly, the process is furthermore particularly suitable for synthesis of compounds of formula VI, which correspond to formula Via

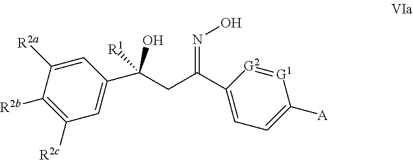

wherein
R¹ is CF₃;
R^{2a} is F, Cl, Br, CF₃, or OCF₃;
R^{2b} and R^{2c} are independently from each other H, F, Cl, Br, CF₃, or OCF₃;
A is A¹, A², or A³; wherein
A¹ is C(═O)N(R⁵)R⁶, C(═O)OR⁹, wherein
A² is

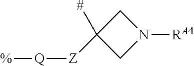

wherein # denotes the bond of group A, and % denotes the bond to G¹;
Q-Z is %-CH₂—O—*, wherein % marks the bond of Q to phenyl, and * the bond of Z to azetidin; and
R^{A4} is H, or C(═O)R^{4A}, wherein
R^{4A} is H, C₁-C₄-alkylcarbonyl, which is unsubstituted or substituted with S(O)ₙ—C₁-C₆-alkyl;
A³ is CH₂—NR⁵C(═O)R⁶;
   G¹, and G² are each CR³, or together form a sulfur atom;
R³ is H or C₁-C₄-alkyl, or two R³ bonded to adjacent carbon atoms may form a five- or sixmembered saturated or aromatic carbocyclic ring, or a dihydrofurane, or
R³ bonded to a carbon atom in position G¹ form a bond to the chain *-Q-Z— in group A²;
R⁵ is H;
R⁶ is H, or C₁-C₆-alkyl which is unsubstituted, or substituted with one or two R⁸;
or R⁵ and R⁶, together with the nitrogen atom to which they are bound, form a 5- or 6-membered saturated, heterocyclic ring, which ring contain 1 or 2 groups selected from O, S, N, and C═O as ring members, which heterocyclic ring is unsubstituted or partially substituted with same or different C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl;
each R⁸ is C(═O)N(R^{10a})R^{10b}, or
two R⁸ present on the same carbon atom of an alkyl group together form ═NOR⁹;
R⁹ being C₁-C₄-alkyl;
R^{10a}, R^{10b} are independently from one another H, C₁-C₆-alkyl, C₁-C₆-haloalkyl, C₂-C₆-alkenyl, C₂-C₆-haloalkenyl, C₂-C₆-alkynyl, C₂-C₆-haloalkynyl.

Particularly preferred are compounds of formula Via, wherein R¹ is CF₃, and the variables have the meanings as shown in Table VI, wherein each compound corresponds to one line.

TABLE VI

| No. | R^{2a},R^{2b},R^{2c} | 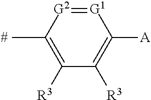 | A |
|---|---|---|---|
| VI-1 | Cl,F,Cl | 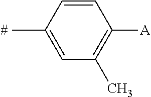 | COOCH₃ |
| VI-2 | Cl,F,Cl | 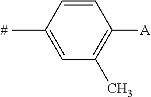 | COOC₂H₅ |
| VI-3 | Cl,H,Cl | 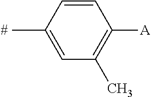 | COOCH₃ |
| VI-4 | Cl,H,Cl | 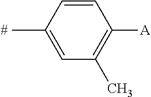 | COOC₂H₅ |
| VI-5 | CF₃,H,Cl | 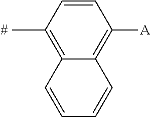 | COOCH₃ |
| VI-6 | CF₃,H,Cl | 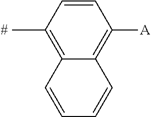 | COOC₂H₅ |
| VI-7 | Cl,Cl,Cl | 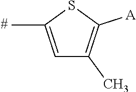 | COOCH₃ |
| VI-8 | Cl,Cl,Cl | 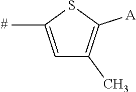 | COOC₂H₅ |
| VI-9 | Cl,F,Cl | 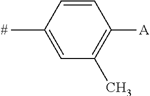 | 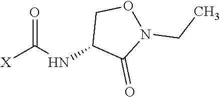 |
| VI-10 | Cl,H,Cl | 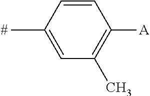 | 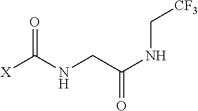 |
| VI-11 | CF₃,H,Cl | 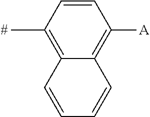 | 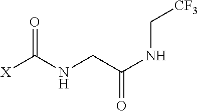 |
| VI-12 | Cl,Cl,Cl | 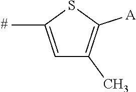 | 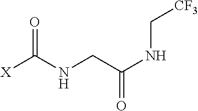 |
| VI-13 | Cl,H,Cl | 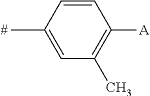 | 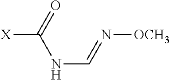 |

| | | |
|---|---|---|
| VI-14 | Cl,F,Cl | 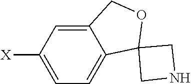 |
| VI-15 | Cl,F,Cl | 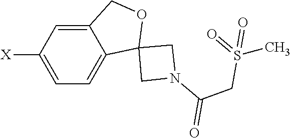 |

In the G¹-G² containing ring: # marks the bond to the oxime group

In group A: X marks the bond to the remainder of the molecule

The following examples illustrate the invention.

### A. PREPARATION EXAMPLES

With appropriate modification of the starting materials, the procedures given in the synthesis description were used to obtain further compounds I. The compounds obtained in this manner are listed in the table that follows, together with physical data.

The products shown below were characterized by melting point determination, by NMR spectroscopy or by the masses ([m/z]) or retention time (RT; [min.]) determined by HPLC-MS or HPLC spectrometry.

HPLC-MS=high performance liquid chromatography-coupled mass spectrometry;

HPLC method A: HPLC method: Phenomenex Kinetex 1.7 μm XB—C18 100A; 50×2.1 mm; mobile phase: A: water+0.1% trifluoroacetic acid (TFA); B: acetonitrile; gradient: 5-100% B in 1.50 minutes; 100% B 0.25 min; flow: 0.8-1.0 ml/min in 1.51 minutes at 60° C. MS: ESI positive, m/z 100-1400.

HPLC method B: HPLC Phenomenex Kinetex 1.7 μm XB—C18 100A, 50×2.1 mm″, Mobile Phase: A: water+0.1% TFA; B:Acetonitrile; Temperature: 60° C.; Gradient: 5% B to 100% B in 1.50 min; 100% B 0.25 min; Flow: 0.8 ml/min to 1.0 ml/min in 1.51 min; MS method: ESI positive; Mass range (m/z): 100-700″.

HPLC method C: Daicel Chiralpak AD-RH 5 μm 150×4.6 mm, mobile phase A: water+0.1% v/v H₃PO₄, B: acetonitrile/2-propanol (1:1) Temperature 50° C., Gradient: 50% B 0 min, 50% B 10 min, 70% B 25 min, 100% B 30 min100% B 35 min, 50% B 35.5 min, total runtime 40 min; flow 1.2 mL/min. UV-detector lambda=216 nm; BW 4 nm; pressure 130 bar.

### Example 1: Preparation of S-fluralaner

### Step 1: preparation of methyl 4-[(3S)-3-(3,5-dichlorophenyl)-4,4,4-trifluoro-3-hydroxy-butanoyl]-2-methyl-benzoate

To a solution of 1-(3,5-dichlorophenyl)-2,2,2-trifluoro-ethanone (preparation known from WO 2010125130, 13.15 g, 54.11 mmol, 1.30 equiv.) in toluene (70 mL) was added R,R-TUC (344 mg, 0.832 mmol, 0.02 equiv.) and methyl 4-acetyl-2-methyl-benzoate (cf. WO 2013/025425, 8.00 g, 41.6 mmol, 1.00 equiv.) and the mixture was stirred at 20 to 25° C. overnight. After completed reaction, all volatiles were removed under reduced pressure and the residue was taken up in petrol ether at 45° C. After 1 h at 20 to 25° C., the precipitate was removed by filtration which contained the racemate of the title compound (5.4 g). The mother liquid was concentrated in vacuum and purified via flash chromatography on silica gel to yield 11.3 g of the title compound (enantiomeric ratio 99.7:0.3).

¹H NMR: (400 MHz, CDCl₃): δ 2.66 (s, 3H), 3.69 (d, 1H), 3.88 (d, 1H), 3.94 (s, 3H), 5.63 (s, 1H), 7.35 (m, 1H), 7.51 (s, 2H), 7.79 (m, 2H), 8.00 (m, 1H) ppm.

Racemate: HPLC Method C: Rt=8.032 min and 8.708 min

Title compound S-isomer: HPLC Method C: Rt=8.094 min

### Step 2: preparation of methyl 4-[(Z)—C-[(2S)-2-(3,5-dichlorophenyl)-3,3,3-trifluoro-2-hydroxy-propyl]-N-hydroxy-carbonimidoyl]-2-methyl-benzoate

To a mixture of methyl 4-[(3S)-3-(3,5-dichlorophenyl)-4,4,4-trifluoro-3-hydroxy-butanoyl]-2-methyl-benzoate (54.4 g, 125 mmol, 1.00 equiv.) and 2,6-lutidine (300 mL) was added solid hydroxylamine hydrochloride (69.49 g, 250 mmol, 2.00 equiv) in several portions at 20 to 25° C. After stirring overnight, the reaction mixture was poured on ice-water (1 L) and extracted with dichloromethane. Combined organic layers were washed with aqueous hydrochloric acid (6N) and water, dried over anhydrous sodium sulfate, filtered and concentrated in vacuum. The residue was triturated with TBME to remove residual lutidine hydrochloride. The filtrate was concentrated and filtered through a plug of silica gel to obtain 46.7 g of the title compound (83% yield).

¹H NMR: (400 MHz, Acetone-d₆): δ 2.49 (s, 3H), 3.39 (d, 1H), 3.70 (d, 1H), 3.88 (s, 3H), 6.04 (s, 1H), 7.10 (s, 1H), 7.15 (d, 1H), 7.36 (s, 1H), 7.48 (s, 2H), 7.76 (d, 1H), 10.38 (s, 1H) ppm.

### Step 3: preparation of methyl 4-[(5S)-5-(3,5-dichlorophenyl)-5-(trifluoromethyl)-4H-isoxazol-3-yl]-2-methyl-benzoate

To a suspension of methyl 4-[(Z)—C-[(2S)-2-(3,5-dichlorophenyl)-3,3,3-trifluoro-2-hydroxy-propyl]-N-hydroxy-carbonimidoyl]-2-methyl-benzoate (5.90 g, 13.1 mmol, 1.00 equiv.) in toluene (30 mL) was added a solution of lithium bis(trimethylsilyl)amide (LiHMDS; 15.7 mL of a 1 M solution in THF, 15.7 mmol, 1.2 equiv.) at 0° C. After 30 min at this temperature, methane sulfonylchloride (3.75 g, 32.8 mmol, 2.5 equiv.) was added slowly and the mixture was allowed to reach 20 to 25° C. After completion of the reaction, water and TBME were added under ice-cooling. The layers were separated, and the organic layer was washed with 1N HCl and water. After drying over Na₂SO₄, the solids were removed by filtration and the filtrate was concentrated in vacuum. The obtained residue was purified via silica gel chromatography to yield 4.20 g of the title compound (74% yield).

¹H NMR: (400 MHz, CDCl₃): δ 2.62 (s, 3H), 3.73 (d, 1H), 3.93 (s, 3H), 4.11 (d, 1H), 7.45 (s, 1H), 7.51-7.58 (m, 3H), 7.96 (d, 1H) ppm.

Racemate: HPLC Method C: Rt=9.544 min and 10.309 min

Title compound S-isomer: HPLC Method C: Rt=9.540 min

### Step 4: preparation of 4-[(5S)-5-(3,5-dichlorophenyl)-5-(trifluoromethyl)-4H-isoxazol-3-yl]-2-methyl-benzoic acid

To a solution of methyl 4-[(5S)-5-(3,5-dichlorophenyl)-5-(trifluoromethyl)-4H-isoxazol-3-yl]-2-methyl-benzoate (21.90 g, 50.67 mmol, 1.00 equiv.) in THF (100 mL) was added an aqueous solution of LiOH (101 mL of a 2 M solution, 101 mmol, 2.0 equiv.) and the mixture was stirred at 40° C. overnight. After complete conversion, the mixture was cooled and aqueous hydrochloric acid (2N) was added to acidify. After extraction with ethyl acetate, the combined organic layers were washed with brine and dried over Na₂SO₄. After filtration and removal of the solvents under reduced pressure, 21.1 g of the title compound (100%) were obtained and used in the next step without purification.

¹H NMR: (400 MHz, CDCl₃): δ 2.69 (s, 3H), 3.73 (d, 1H), 4.12 (d, 1H), 7.44 (s, 1H), 7.52 (s, 2H), 7.65 (m, 2H), 8.12 (d, 1H) ppm.

### Step 5: preparation of 4-[(5S)-5-(3,5-dichlorophenyl)-5-(trifluoromethyl)-4H-isoxazol-3-yl]-2-methyl-N-[2-oxo-2-(2,2,2-trifluoroethylamino)ethyl]benzamide (S-fluralaner)

To a mixture of 4-[(5S)-5-(3,5-dichlorophenyl)-5-(trifluoromethyl)-4H-isoxazol-3-yl]-2-methyl-benzoic acid (200 mg, 478 mmol, 1.00 equiv.), [2-oxo-2-(2,2,2-trifluoroethylamino)ethyl]ammonium chloride (111 mg, 574 mmol, 1.20 equiv.) and bromotripyrrolidinophosphonium hexafluoro-phosphate (PyBroP®; 267 mg, 574 mmol, 1.00 equiv.) in dichloromethane (10 mL) was added diisopropylethylamine (0.34 mL, 0.26 g, 2.0 mmol, 4.2 euqiv.) at 0-5° C. and the mixture was allowed to reach 20 to 25° C. After completion of the reaction, all volatiles were removed in vacuum and the residue was taken up in ethyl acetate. This solution was washed with 2N HCLI and water before being dried over Na₂SO₄. After filtration, the solvents were removed in vacuum and the residue was purified via flash chromatography on silica gel to obtain 154 mg S-fluralaner (55%) as a colorless solid.

LC-MS: Mass calculated for C₂₂H₁₇Cl₂F₆N₃O₃⁺ [(M+H)⁺)] 556.3, found 556.1; RT=1.311 min (Method A).

¹H NMR: (400 MHz, CDCl₃): δ 2.49 (s, 3H), 3.73 (d, 1H), 3.95 (m, 2H), 4.09 (d, 1H), 4.21 (d, 2H), 6.68 (m, 1H), 6.86 (m, 1H), 7.39-7.61 (m, 6H) ppm.

### Example 2: Preparation of S-isocycloseram

### Step 1: preparation of methyl 4-[(3S)-3-(3,5-dichloro-4-fluoro-phenyl)-4,4,4-trifluoro-3-hydroxy-butanoyl]-2-methyl-benzoate

The reaction was performed analogously to Example 1, step 1. Crystallization of the crude mixture from petrol ether gave the title compound in the mother liquor with enantiomeric ratio (S:R) of 98:2 in isolated yield of 61%.

¹H-NMR (400 MHz, CDCl₃): δ=2.67 (s, 3H), 3.71 (d, 1H), 3.85 (d, 1H), 3.96 (s, 3H), 5.69 (s, 1H), 7.58 (d, 2H), 7.78 (m, 2H), 8.00 (d, 1H)

### Step 2: preparation of methyl 4-[(Z)—C-[(2S)-2-(3,5-dichloro-4-fluoro-phenyl)-3,3,3-trifluoro-2-hydroxy-propyl]-N-hydroxy-carbonimidoyl]-2-methyl-benzoate

To a 2 L four necked flask with propeller stirring, 2,6-lutidine (1250 mL) and hydroxylammonium chloride (69.00 g, 3 equiv.) were added. After 1 h at 20-25° C., methyl 4-[(3S)-3-(3,5-di-chloro-4-fluoro-phenyl)-4,4,4-trifluoro-3-hydroxy-butanoyl]-2-methyl-benzoate (150 g, 1 equiv.) in 2,6-luditine (250 mL) were added. After the starting material was consumed, the reaction mixture was added to ice water (1.5 L) and ethyl acetate (1 L) and the organic layer was separated. The aqueous layer was extracted with ethyl acetate (2×500 mL) and combined organic layers were washed with ice-cold 6N HCl (1.7 L). After washing with water (2×1.5 L), the organic layer was dried over Na₂SO₄, filtered and evaporated to dryness. The resulting residue was triturated with n-pentane (150 mL) to yield the title compound (154 g, 99%).

¹H-NMR (400 MHz, acetone-d₆): δ=2.49 (s, 3H), 3.36 (d, 1H), 3.71 (d, 1H), 3.87 (s, 3H), 6.10 (s, 1H), 7.09 (s, 1H), 7.16 (d, 1H), 7.56 (d, 2H), 7.77 (d, 1H), 10.36 (s, 1H).

### Step 3: preparation of methyl 4-[(5S)-5-(3,5-dichloro-4-fluoro-phenyl)-5-(trifluoromethyl)-4H-isoxazol-3-yl]-2-methyl-benzoate

To a solution of methyl 4-[(Z)—C-[(2S)-2-(3,5-dichloro-4-fluoro-phenyl)-3,3,3-trifluoro-2-hydroxy-propyl]-N-hydroxy-carbonimidoyl]-2-methyl-benzoate (120.6 g, 1.0 equiv.) in THF (600 mL) was added a solution of LiHMDS (566 mL of a 1M solution in THF, 2.2 equiv.) at −5° C. After 30 min at that temperature, a solution of mesyl chloride (42.3 mL, 62.5 g, 2.12 equiv. in 100 mL THF) was added slowly, whereupon the temperature rose to 0° C. After 2 h at 0° C., the cooling bath was removed, and the mixture was stirred at 20-25° C. overnight. Water (200 mL) and 2N HCl (200 mL) were added at 0-5° C., and the volatiles were removed in vacuum. The remaining aqueous mixture was extracted with ethyl acetate. Combined organic layers were washed with water and dried over Na₂SO₄. After removal of the solvents, the residue was purified via flash chromatography on silica gel to yield the title compound (90.0 g, 78%).

¹H-NMR (400 MHz, CDCl₃): δ=2.63 (s, 3H), 3.69 (d, 1H), 3.89 (s, 3H), 4.11 (d, 1H), 7.52 (m, 2H), 7.60 (m, 2H), 7.96 (d, 1H)

### Step 4: preparation of 4-[(5S)-5-(3,5-dichloro-4-fluoro-phenyl)-5-(trifluoromethyl)-4H-isoxazol-3-yl]-2-methyl-benzoic acid

To a solution of methyl 4-[(5S)-5-(3,5-dichloro-4-fluoro-phenyl)-5-(trifluoromethyl)-4H-isoxazol-3-yl]-2-methyl-benzoate (150 g, 333 mmol, 1.00 equiv.) in THF (1500 mL) was added aq. 1M LiOH (666 mL) at 20-25° C. The reaction mixture was stirred at 40° C. over night, cooled to 0° C. and 2M HCl was added until pH 1 was reached. The organic layer was separated and the aqueous layer was extracted with ethyl acetate (2×500 mL). Combined organic layers were washed with brine and dried over Na₂SO₄. Removal of the solvents in vacuum yielded the title compound (145 g, 100%).

¹H-NMR (400 MHz, CDCl₃): δ=2.71 (s, 3H), 3.72 (d, 1H), 4.11 (d, 1H), 7.54-7.63 (m, 4H), 8.10 (m, 1H).

### Step 5: preparation of 4-[(5S)-5-(3,5-dichloro-4-fluoro-phenyl)-5-(trifluoromethyl)-4H-isoxazol-3-yl]-2-methyl-benzoyl chloride

To a solution of 4-[(5S)-5-(3,5-dichloro-4-fluoro-phenyl)-5-(trifluoromethyl)-4H-isoxazol-3-yl]-2-methyl-benzoic acid (160 g, 366 mmol, 1.00 equiv.) in dichloromethane (1700 mL) was added DMF (1 mL), followed by oxalyl chloride (94 mL, 1.10 mol, 3 equiv.) in dichloromethane (300 mL) at 20-25° C. within 3 h. After completion of the reaction, all volatiles were removed in vacuum to yield the title compound (166 g, 100%).

¹H-NMR (400 MHz, CDCl₃): δ=2.60 (s, 3H), 3.71 (d, 1H), 4.11 (d, 1H), 7.54-7.70 (m, 4H), 8.27 (m, 1H).

### Step 6: preparation of 4-[(5S)-5-(3,5-dichlorophenyl)-5-(trifluoromethyl)-4H-isoxazol-3-yl]-2-methyl-N-[2-oxo-2-(2,2,2-trifluoroethylamino)ethyl]benzamide (S-isocycloseram)

A suspension of [(4R)-2-ethyl-3-oxo-isoxazolidin-4-yl]ammoniumchloride (67.12 g, 403.3 mmol (1.100 equiv.) in THF (1500 mL) was added DMAP (2.24 g, 0.018 mol, 0.05 equiv.), followed by diisopropyl ethylamine (251 mL, 1.83 mol, 5 equiv.) at 0-5° C. within 60 min. After 30 min at 0-5° C., a solution of 4-[(5S)-5-(3,5-dichloro-4-fluoro-phenyl)-5-(trifluoromethyl)-4H-isoxazol-3-yl]-2-methyl-benzoyl chloride (166 g, 366 mmol, 1.00 equiv.) in THF (400 mL) was added at 0°−5° C. within 120 min. The cooling bath was removed, and the mixture was stirred overnight whereupon the product has formed and no starting material could be observed. To this mixture, 2M HCl was added until pH 1 was reached. The layers were separated, and the aqueous layer was extracted with ethyl acetate (2×). Combined organic layers were washed with water and brine, then dried over Na₂SO₄. After removal of the solvents, the residue was triturated with diisopropyl ether to yield the title compound (166.6 g, 83%) as a colorless solid (mp. 141° C.).

¹H-NMR (400 MHz, CDCl₃): δ=1.25 (t, 3H), 2.49 (s, 3H), 3.56-3.77 (m, 3H), 4.01-4.12 (m, 2H), 4.85 (m, 1H), 4.99 (m, 1H), 6.47 (m, 1H), 7.46-7.62 (m, 5H).

### Example 3: Preparation of S-sarolaner

### Step 1: Synthesis of (3S)-3-(3,5-dichloro-4-fluoro-phenyl)-4,4,4-trifluoro-3-hydroxy-1-[1′-(2-me-thylsulfonylacetyl)spiro[3H-isobenzofuran-1,3′-azetidine]-5-yl]butan-1-one

The reaction was performed analogously to Example 1, step 1. Crystallization of the crude mixture from petrol ether gave the title compound in the mother liquor with enantiomeric ratio of 99:1.

¹H-NMR (400 MHz, CDCl₃): δ=3.20 (s, 3H), 3.71 (d, 1H), 3.82 (d, 1H), 3.85 (s, 2H), 4.36 (d, 1H), 4.47 (d, 1H), 5.19 (s, 1H), 5.70 (d, 1H), 7.55 (d, 2H), 7.70 (d, 1H), 7.79 (s, 1H), 8.00 (d, 1H).

### Step 2: Synthesis of 1-[6-[(Z)—C-[(2S)-2-(3,5-dichloro-4-fluoro-phenyl)-3,3,3-trifluoro-2-hydroxy-propyl]-N-hydroxy-carbonimidoyl]spiro[1H-isobenzofuran-3,3′-azetidine]-1′-yl]-2-methylsulfonyl-ethanone

To a mixture of hydroxylamine hydrochloride (95 mg) and 2,6-lutidine (10 mL) was added a solution of 3S)-3-(3,5-dichloro-4-fluoro-phenyl)-4,4,4-trifluoro-3-hydroxy-1-[1′-(2-methylsulfonylacetyl)spiro[3H-isobenzofuran-1,3′-azetidine]-5-yl]butan-1-one (160 mg) in 2,6-lutidine (5 mL). After the starting material was consumed, the reaction mixture was added to ethyl acetate and washed with 1M HCl (3×), water (2×), before the organic layer was dried over Na₂SO₄. After removal of all volatiles in vacuum the title compound (160 mg) was obtained as a colorless solid. that was used in the next stage without further purification.

### Step 3: Synthesis of 1-[6-[(5S)-5-(3,5-dichloro-4-fluoro-phenyl)-5-(trifluoromethyl)-4H-isoxazol-3-yl]spiro[1H-isobenzofuran-3,3′-azetidine]-1′-yl]-2-methylsulfonyl-ethanone (S-sarolaner)

To a solution of 1-[6-[(Z)—C-[(2S)-2-(3,5-dichloro-4-fluoro-phenyl)-3,3,3-trifluoro-2-hydroxy-propyl]-N-hydroxy-carbonimidoyl]spiro[1H-isobenzofuran-3,3′-azetidine]-1′-yl]-2-methylsulfonyl-ethanone (160 mg, 267 mmol) in THF (10 mL) was added LiHMDS (0.59 mL of a 1 M solution, 587 mmol, 2.2 equiv.) at 0° C. The mixture was stirred at 0° C. for 60 min, then mesyl chloride (61 mg, 534 mmol, 2.0 equiv.) was added drop wise. The mixture was allowed to reach 20-25° C. and stirred for 12 h. TLC showed completion of the reaction, and ethyl acetate and aqueous NH₄Cl solution were added. The layers were separated and the organic layer was washed with water (3×). After removal of the solvents, the residue was purified via flash chromatography on silica gel to obtain the title compound (101 mg, 65%).

¹H-NMR (400 MHz, DMSO-d₆): δ=3.13 (s, 3H), 4.19-4.24 (m, 4H), 4.30-4.42 (m, 2H), 4.59 (s, 2H), 5.17 (s, 2H), 7.64-7.83 (m, 5H).

### Example 4: Preparation of 4-[(5S)-5-[3-chloro-5-(trifluoromethyl)phenyl]-5-(trifluoromethyl)-4H-isoxazol-3-yl]-N-[2-oxo-2-(2,2,2-trifluoroethylamino)ethyl]naphthalene-1-carboxamide (S-afoxolaner)

### Step 1: Preparation of methyl 4-[(3S)-3-[3-chloro-5-(trifluoromethyl)phenyl]-4,4,4-trifluoro-3-hydroxy-butanoyl]naphthalene-1-carboxylate

The reaction was performed analogously to Example 1, step 1. Crystallization of the crude mixture from petrol ether gave the title compound in the mother liquor with enantiomeric ratio of 96:4.

The subsequent steps 2 to 5 were performed in analogy to Example 3.

¹H-NMR of 4-[(5S)-5-[3-chloro-5-(trifluoromethyl)phenyl]-5-(trifluoromethyl)-4H-isoxazol-3-yl]-N-[2-oxo-2-(2,2,2-trifluoroethylamino)ethyl]naphthalene-1-carboxamide (S-afoxolaner) (500 MHz, CDCl₃): δ=3.85-3.99 (m, 3H), 4.23-4.36 (m, 3H), 7.29-7.64 (m, 6H), 7.71 (s, 1H), 7.83 (s, 1H), 7.89 (s, 1H), 8.20 (m, 1H), 8.81 (m, 1H).

1. A process for preparing compounds a compound of formula I 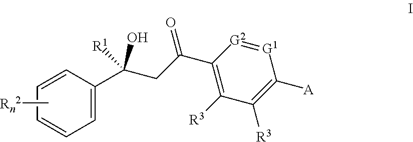
wherein
R¹ is halomethyl;
each R² is independently H, halogen, CN, N₃, NO₂, SCN, SF₅, C₁-C₆-alkyl, C₃-C₈-cycloalkyl, C₂-C₆-alkenyl, C₂-C₆-alkynyl, which groups are unsubstituted, partially or fully halogenated and/or substituted with one or more same or different R⁸, Si(R¹²)₃, OR⁹, S(O)ₙR⁹, NR^{10a}R^{10b},
phenyl which is unsubstituted or partially or fully substituted with R¹¹, and a 3- to 10-membered saturated, partially or fully unsaturated heteromonocyclic or heterobicyclic ring containing 1, 2, 3 or 4 heteroatoms N, O, and/or S as ring members, which ring is unsubstituted, or substituted with one or more same or different R¹¹, preferably the unsubstituted or substituted HET;
n is 0, 1, or 2;
G¹, G² are each CR³, or together form a sulfur atom;
each R³ is independently selected from the meanings mentioned for R², or two R³ bonded to adjacent carbon atoms may form a five- or six-membered saturated, partially or fully unsaturated carbocyclic ring, or a dihydrofurane, or
R³ bonded to carbon atom in position G¹ form a bond to the chain *-Q-Z— in group A²;
A is a group A¹, A², A³, or A⁴; wherein
A¹ is C(═W)Y;
W is O, or S;
Y is N(R⁵)R⁶, or OR⁹;
A² is 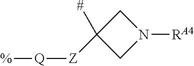
wherein # denotes the bond of group A, and % denotes the bond to G¹;
Q-Z is %-CH₂—O—*, ′%-CH₂—S(O)ₙ—*, or %-C(═O)—O—*, wherein % marks the bond of Q to phenyl, and * the bond of Z to azetidin; and
R^{A4} is H or C(═O)R^{4A}, wherein
R^{4A} is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₄-alkylcarbonyl, which aliphatic groups are unsubstituted or substituted with one or more radicals R⁴¹;
C₃-C₆-cycloalkyl, C₃-C₆-halocycloalkyl which cyclic groups are unsubstituted or substituted with one or more R⁴²;
C(═O)N(R⁴³)R⁴⁴, N(R⁴³)R⁴⁵, CH═NOR⁴⁶;
phenyl, heterocycle, or hetaryl HET which rings are unsubstituted or partially or fully substituted with R^{A};
R⁴¹ is independently OH, CN, C₁-C₆-alkoxy, C₁-C₆-haloalkoxy, S(O)ₙ—C₁-C₆-alkyl, S(O)ₙ—C₁-C₆-haloalkyl, C(═O)N(R⁴³)R⁴⁴,
C₃-C₆-cycloalkyl, or C₃-C₆-halocycloalkyl which cycles are unsubstituted or substituted with one or more R⁴¹¹; or
phenyl, heterocycle or hetaryl HET which rings are unsubstituted or partially or fully substituted with R^{A};
R⁴¹¹ is independently OH, CN, C₁-C₂-alkyl, or C₁-C₂-haloalkyl;
R⁴³ is H, or C₁-C₆-alkyl,
R⁴⁴ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, or C₃-C₆-cycloalkyl, C₃-C₆-halocycloalkyl, C₃-C₆-cycloalkylmethyl, or C₃-C₆-halocycloalkylmethyl which rings are unsubstituted or substituted with a cyano;
R⁴⁵ H, C₁-C₆-alkyl, C₁-C₆-haloalkyl, C₂-C₄-alkenyl, C₂-C₄-alkynyl, CH₂—CN, C₃-C₆-cycloalkyl, C₃-C₆-halocycloalkyl, C₃-C₆-cycloalkylmethyl, C₃-C₆-halocycloalkylmethyl, phenyl and hetaryl HET which aromatic rings are unsubstituted or partially or fully substituted with R^{A};
R⁴² C₁-C₆-alkyl, C₁-C₆-haloalkyl, or a group as defined for R⁴¹;
R⁴⁶ is independently H, C₁-C₆-alkyl, or C₁-C₆-haloalkyl;
R^{A} is independently selected from halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, S(O)ₙ—C₁-C₄-alkyl, S(O)ₙ—C₁-C₄-haloalkyl, C₁-C₄-alkylcarbonyl, C₁-C₄-haloalkylcarbonyl, C(═O)N(R⁴³)R⁴⁴; or
two R^{A} present on the same carbon atom of a saturated or partially saturated ring may form together ═O or ═S; or
two R^{A} present on the same S or SO ring member of a heterocyclic ring may together form a group ═N(C₁-C₆-alkyl), ═NO(C₁-C₆-alkyl), ═NN(H)(C₁-C₆-alkyl) or ═NN(C₁-C₆-alkyl)₂;
A³ is CH₂—NR⁵C(═W)R⁶;
A⁴ is cyano;
R⁵ is independently selected from the meanings mentioned for R²;
R⁶ is H, CN, C₁-C₁₀-alkyl, C₃-C₈-cycloalkyl, C₂-C₁₀-alkenyl, C₂-C₁₀-alkynyl, which groups are unsubstituted, partially or fully halogenated and/or substituted with one or more same or different R⁸; or
S(O)ₙR⁹, or C(═O)R⁸; or
a 3- to 8-membered saturated, partially or fully unsaturated heterocyclic ring, which ring may contain 1, 2, 3, or 4 heteroatoms O, S, N, C═O and/or C═S as ring members, which heterocyclic ring is unsubstituted or partially or fully substituted with same or different halogen, CN, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, which groups are unsubstituted, or partially or fully substituted with same or different R⁸, or phenyl which may be partially or fully substituted with R¹¹;
or R⁵ and R⁶ together form a group ═C(R⁸)₂, ═S(O)ₘ(R⁹)₂, ═NR^{10a}, or ═NOR⁹;
R^{7a}, R^{7b} are each independently H, halogen, CN, C₁-C₆-alkyl, C₃-C₈-cycloalkyl, C₂-C₆-alkenyl, or C₂-C₆-alkynyl, which groups are unsubstituted, partially or fully halogenated and/or substituted with same or different R⁸;
each R⁸ is independently CN, N₃, NO₂, SCN, SF₅, C₃-C₈-cycloalkyl, C₃-C₈-halocycloalkyl, wherein the carbon chains may be substituted with one or more R¹³;
Si(R¹²)₃, OR⁹, OSO₂R⁹, S(O)ₙR⁹, N(R^{10a})R^{10b}, C(═O)N(R^{10a})R^{10b}, C(═S)N(R^{10a})R^{10b}, C(═O)OR⁹, CH═NOR⁹,
phenyl, which is unsubstituted or partially or fully substituted with same or different R¹⁶, or
a 3-, 4-, 5-, 6- or 7-membered saturated, partially or fully unsaturated heterocyclic ring comprising 1, 2 or 3 heteroatoms N, O, and/or S as ring members, which ring is unsubstituted or partially or fully substituted with same or different R¹⁶, or
two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b}; or
two radicals R⁸, together with the carbon atoms of the alkyl, alkenyl, alkynyl or cycloalkyl group which they are bonded to, form a 3-, 4-, 5-, 6-, 7- or 8-membered saturated or partially unsaturated carbocyclic or heterocyclic ring, which heterocyclic ring comprises 1, 2, 3 or 4 heteroatoms N, O, and/or S as ring members, and which ring is unsubstituted, or partially or fully substituted with same or different R¹⁶; and
R⁸ as a substituent on a cycloalkyl ring may additionally be C₁-C₆-alkyl, C₁-C₆-haloalkyl, C₂-C₆-alkenyl, C₂-C₆-haloalkenyl, C₂-C₆-alkynyl, and C₂-C₆-haloalkynyl, which groups are unsubstituted, or partially or fully substituted with same or different R¹³; and
R⁸ in the groups C(═O)R⁸ and ═C(R⁸)₂ may additionally be H, halogen, C₁-C₆-alkyl, C₁-C₆-haloalkyl, C₂-C₆-alkenyl, C₂-C₆-haloalkenyl, C₂-C₆-alkynyl, or C₂-C₆-haloalkynyl, which groups are unsubstituted, or partially or fully substituted with same or different R¹³;
each R⁹ is independently H, CN, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, or C₂-C₆-haloalkynyl, which groups are unsubstituted, or partially or fully substituted with same or different R¹³, or C₁-C₆-alkyl-C(═O)OR¹⁵, C₁-C₆-alkyl-C(═O)N(R^{14a})R^{14b}, C₁-C₆-alkyl-C(═S)N(R^{14a})R^{14b}, C₁-C₆-alkyl-C(═NR¹⁴)N(R^{14a})R^{14b}, Si(R¹²)₃, S(O)ₙR¹⁵, S(O)ₙN(R^{14a})R^{14b}, N(R^{10a})R^{10b}, N═C(R¹³)₂, C(═O)R¹³, C(═O)N(R^{14a})R^{14b}, C(═S)N(R^{14a})R^{14b}, C(═O)OR¹⁵, or
phenyl, which is unsubstituted, or partially or fully substituted with R¹⁶; and
a 3- to 7-membered saturated, partially or fully unsaturated heterocyclic ring comprising 1, 2 or 3 heteroatoms N, O, and/or S as ring members, which ring is unsubstituted, or partially or fully substituted with same or different R¹⁶; and
R⁹ in the groups S(O)ₙR⁹ and OSO₂R⁹ may additionally be C₁-C₆-alkoxy, or C₁-C₆-haloalkoxy;
R^{10a}, R^{10b} are independently from one another H, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂—C₆-alkynyl, C₂-C₆-haloalkynyl, which groups are unsubstituted, or partially or fully substituted with same or different R¹³;
C₁-C₆-alkyl-C(═O)OR¹⁵, C₁-C₆-alkyl-C(═O)N(R^{14a})R^{14b}, C₁-C₆-alkyl-C(═S)N(R^{14a})R^{14b},
C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio, S(O)ₙR¹⁵, S(O)ₙN(R^{14a})R^{14b}, C(═O)R¹³, C(═O)OR¹⁵, C(═O)N(R^{14a})R^{14b}, C(═S)R¹³, C(═S)SR¹⁵, C(═S)N(R^{14a})R^{14b}, C(═NR¹⁴)R¹³;
phenyl, which is unsubstituted, or partially or fully substituted with same or different R¹⁶; and
a 3-, 4-, 5-, 6- or 7-membered saturated, partially or fully unsaturated heterocyclic ring comprising 1, 2, 3 or 4 heteroatoms N, O, and/or S as ring members, which ring is unsubstituted, or partially or fully substituted with same or different R¹⁶, preferably unsubstituted or substituted HET; or
R^{10a} and R^{10b} together with the nitrogen atom they are bonded to form a 3- to 8-membered saturated, partially or fully unsaturated heterocyclic ring, which ring may additionally contain one or two heteroatoms N, O, and/or S as ring members, which ring is unsubstituted, or partially or fully substituted with same or different halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be partially or fully substituted with R¹⁶, and a 3-, 4-, 5-, 6,- or 7-membered saturated, partially or fully unsaturated heterocyclic ring comprising 1, 2 or 3 heteroatoms N, O, and/or S as ring members, which ring is unsubstituted, or partially or fully substituted with same or different R¹⁶; or
R^{10a} and R^{10b} together form a group ═C(R¹³)₂, ═S(O)ₘ(R¹⁵)₂, ═S(O)ₘR¹⁵N(R^{14a})R^{14b}, ═NR¹⁴, or ═NOR¹⁵;
R¹¹ is halogen, CN, N₃, NO₂, SCN, SF₅, C₁-C₁₀-alkyl, C₃-C₈-cycloalkyl, C₂-C₁₀-alkenyl, C₂-C₁₀-alkynyl, which groups are unsubstituted, partially or fully halogenated, and/or may be substituted with same or different R⁸, or
OR⁹, NR^{10a}R^{10b}, S(O)nR9, Si(R¹²)₃;
phenyl, which is unsubstituted, or partially or fully substituted with same or different R¹⁶; and
a 3- to 7-membered saturated, partially or fully unsaturated aromatic heterocyclic ring comprising 1, 2, 3, or 4 heteroatoms N, O, and/or S as ring members, which ring is unsubstituted, or partially or fully substituted with same or different R¹⁶; or
two R¹¹ present on the same ring carbon atom of an unsaturated or partially unsaturated heterocyclic ring may together form a group ═O, ═C(R¹³)₂, ═S, ═S(O)ₘ(R¹⁵)₂, ═S(O)ₘR¹⁵N(R^{14a})R^{14b}, ═NR¹⁴, ═NOR¹⁵, or ═NN(R^{14a})R^{14b};
or two R¹¹ bound on adjacent ring atoms form together with the ring atoms to which they are bound a saturated 3- to 9-membered ring, which ring may contain 1 or 2 heteroatoms O, S, N, and/or NR¹⁴, and/or 1 or 2 groups C═O, C═S, C═NR¹⁴ as ring members, and which ring is unsubstituted, or partially or fully substituted with same or different halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be partially or fully substituted with same or different R¹⁶, and a 3- to 7-membered saturated, partially or fully unsaturated heterocyclic ring containing 1, 2, or 3 heteroatoms N, O, and/or S as ring members, which ring is unsubstituted, or partially or fully substituted with same or different R¹⁶;
each R¹² is independently C₁-C₄-alkyl and phenyl, which is unsubstituted, or partially or fully substituted with same or different C₁-C₄-alkyl;
each R¹³ is independently CN, NO₂, OH, SH, SCN, SF₅, C₁-C₆-alkoxy, C₁-C₆-haloalkoxy, SOₙ—C₁-C₆-alkyl, SOₙ—C₁-C₆-haloalkyl, Si(R¹²)₃, —C(═O)N(R^{14a})R^{14b}, C₃-C₈-cycloalkyl which is unsubstituted, partially or fully halogenated or substituted with 1 or 2 same or different C₁-C₄-alkyl, C₃-C₄-cycloalkyl, C₁-C₄-alkoxy, C₁-C₄-haloalkoxy and/or oxo; phenyl, benzyl, phenoxy, where the phenyl moiety may be substituted with one or more same or different R¹⁶; and
a 3- to 7-membered saturated, partially or fully unsaturated heterocyclic ring containing 1, 2, or 3 heteroatoms N, O, and/or S, as ring members, which ring is unsubstituted, or partially or fully substituted with same or different R¹⁶; or
two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═N(C₁-C₆-alkyl) or ═NO(C₁-C₆-alkyl); and
R¹³ as a substituent of a cycloalkyl ring may additionally be C₁-C₆-alkyl, C₂-C₆-alkenyl or C₂-C₆-alkynyl, which groups are unsubstituted, partially or fully halogenated, or substituted with 1 or 2 CN, C₃-C₄-cycloalkyl, C₁-C₄-alkoxy, C₁-C₄-haloalkoxy, and oxo; and
R¹³ in groups ═C(R¹³)₂, N═C(R¹³)₂, C(═O)R¹³, C(═S)R¹³, and C(═NR¹⁴)R¹³ may additionally be H, halogen, C₁-C₆-alkyl, C₂-C₆-alkenyl, or C₂-C₆-alkynyl, which groups are unsubstituted, partially or fully halogenated, or substituted with 1 or 2 CN, C₃-C₄-cycloalkyl, C₁-C₄-alkoxy, C₁-C₄-haloalkoxy, and oxo;
each R¹⁴ is independently H, CN, C₁-C₆-alkoxy, C₁-C₆-haloalkoxy, SOₙ—C₁-C₆-alkyl, SOₙ—C₁-C₆-haloalkyl, Si(R¹²)₃;
C₁-C₆-alkyl, C₂-C₆-alkenyl, C₂-C₆-alkynyl, which groups are unsubstituted, partially or fully halogenated, or substituted with 1 or 2 CN, C₁-C₄-alkoxy, C₁-C₄-haloalkoxy, SOₙ—C₁-C₄-alkyl, C₃-C₆-cycloalkyl which is unsubstituted or substituted with 1 or 2 substituents halogen and CN;
and oxo;
C₃-C₈-cycloalkyl which is unsubstituted, or partially or fully halogenated or substituted with 1 or 2 CN, C₁-C₄-alkyl, C₁-C₄-alkoxy, C₁-C₄-haloalkoxy, SOₙ—C₁-C₆-alkyl, C₃-C₄-cycloalkyl, C₃-C₄-cycloalkyl-C₁-C₄-alkyl-, which groups are unsubstituted, or substituted with 1 or 2 substituents selected from halogen and CN;
phenyl, benzyl, pyridyl, phenoxy, which cyclic moieties are unsubstituted, or substituted with one or more same or different halogen, CN, NO₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, and C₁-C₆-alkoxycarbonyl;
and a 3-, 4-, 5- or 6-membered saturated, partially or fully unsaturated heterocyclic ring comprising 1, 2 or 3 heteroatoms N, O, and/or S as ring members, which ring is unsubstituted, or partially or fully substituted with same or different R¹⁶;
R^{14a} and R^{14b} independently of each other, have one of the meanings given for R¹⁴; or
R^{14a} and R^{14b}, together with the nitrogen atom to which they are bound, form a 3- to 7-membered saturated, partially, or fully unsaturated heterocyclic ring, wherein the ring may additionally contain 1 or 2 heteroatoms N, O, and/or S as ring members, which ring is unsubstituted, or partially or fully substituted with same or different halogen, C₁-C₄-alkyl, C₁-C₄-haloalkyl, C₁-C₄-alkoxy, or C₁-C₄-haloalkoxy; or
R^{14a} and R¹⁴ or R^{14b} and R¹⁴, together with the nitrogen atoms to which they are bound in the group C(═NR¹⁴)N(R^{14a})R^{14b}, form a 3- to 7-membered partially, or fully unsaturated heterocyclic ring, wherein the ring may additionally contain 1 or 2 heteroatoms N, O, and/or S as ring members, which ring is unsubstituted, or partially or fully substituted with same or different halogen, C₁-C₄-haloalkyl, C₁-C₄-alkoxy, or C₁-C₄-haloalkoxy;
each R¹⁵ is independently H, CN, Si(R¹²)₃
C₁-C₆-alkyl, C₂-C₆-alkenyl, C₂-C₆-alkynyl, which groups are unsubstituted, partially or fully halogenated, or substituted with 1 or 2 radicals C₃-C₄-cycloalkyl, C₁-C₄-alkoxy, C₁-C₄-haloalkoxy, SOₙ—C₁-C₆-alkyl, or oxo;
C₃-C₈-cycloalkyl which is unsubstituted, partially or fully halogenated or substituted with 1 or 2 radicals C₁-C₄-alkyl, C₃-C₄-cycloalkyl, C₁-C₄-alkoxy, C₁-C₄-haloalkoxy, SOₙ—C₁-C₆-alkyl, or oxo;
phenyl, benzyl, pyridyl, and phenoxy, which rings are unsubstituted, partially or fully halogenated, or substituted with 1, 2 or 3 substituents C₁-C₆-alkyl, C₁-C₆-haloalkyl, C₁-C₆-alkoxy, C₁-C₆-haloalkoxy, or (C₁-C₆-alkoxy)carbonyl;
each R¹⁶ is independently halogen, NO₂, CN, OH, SH, C₁-C₆-alkoxy, C₁-C₆-haloalkoxy, SOₙ—C₁-C₆-alkyl, SOₙ—C₁-C₆-haloalkyl, C₁-C₄-alkylcarbonyl, C₁-C₄-haloalkylcarbonyl, C₁-C₄-alkoxycarbonyl, C₁-C₄-haloalkoxycarbonyl, aminocarbonyl, C₁-C₄-alkylaminocarbonyl, di-(C₁-C₄-alkyl)-aminocarbonyl, Si(R¹²)₃;
C₁-C₆-alkyl, C₂-C₆-alkenyl, C₂-C₆-alkynyl, which groups are unsubstituted, partially or fully halogenated, or substituted with 1 or 2 radicals CN, C₃-C₄-cycloalkyl, C₁-C₄-alkoxy, C₁-C₄-haloalkoxy, or oxo;
C₃-C₈-cycloalkyl which is unsubstituted, partially or fully halogenated or substituted with 1 or 2 radicals CN, C₁-C₄-alkyl, C₃-C₄-cycloalkyl, C₁-C₄-alkoxy, C₁-C₄-haloalkoxy, or oxo;
phenyl, benzyl, pyridyl and phenoxy, which rings are unsubstituted, partially or fully halogenated, or substituted with 1, 2 or 3 substituents C₁-C₆-alkyl, C₁-C₆-haloalkyl, C₁-C₆-alkoxy, C₁-C₆-haloalkoxy, or (C₁-C₆-alkoxy)carbonyl; or
two R¹⁶ present together on the same atom of an unsaturated or partially unsaturated ring may be ═O, ═S, ═N(C₁-C₆-alkyl), ═NO—C₁-C₆-alkyl, ═CH(C₁-C₄-alkyl), or ═C(C₁-C₄-alkyl)₂; or
two R¹⁶ on two adjacent carbon atoms form together with the carbon atoms they are bonded to a 4- to 8-membered saturated, partially or fully unsaturated ring, wherein the ring may contain 1 or 2 heteroatoms N, O, and/or S as ring members, which ring is unsubstituted, or partially or fully substituted with same or different halogen, C₁-C₄-alkyl, C₁-C₄-haloalkyl, C₁-C₄-alkoxy, or C₁-C₄-haloalkoxy;
each n is independently 0, 1, or 2; and
each m is independently 0, or 1;
wherein the shown enantiomer has at least 50% ee;
by condensation of a ketone of formula II with an acetyl compound of formula III, 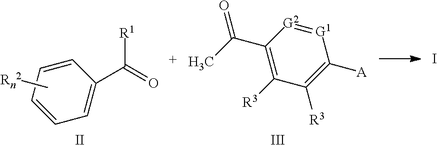
wherein the variables have the meanings given for formula I,
in the presence of a catalyst of formula IV 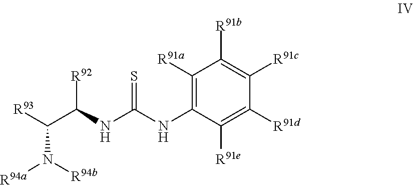
wherein the variables have following meanings:
in case IVa:
R^{91a} to R^{91e} are independently from one another selected from H, CN, NO₂, and C₁-C₆-alkoxycarbonyl;
R⁹² and R⁹³ together with the carbon atoms they are bound to form a cyclohexyl ring;
R^{94a}, R^{94b} are selected from C₁-C₃-alkyl;
in case IVb:
R^{91a} to R^{91e} are independently from one another selected from H, CN, NO₂, and C₁-C₆-alkoxycarbonyl;
R⁹² is selected from 6-methoxy-4-quinolyl, and 4-quinolyl;
R⁹³, R^{94a} and R^{94b} together with the bridging nitrogen atom form a bridged ring system containing 5 to 10 ring members which is unsubstituted or substituted with one or more halogen, C₁-C₄-alkyl, C₁-C₂-haloalkyl, or C₂-C₄-alkenyl, wherein two substitutents bound to the same C-atom may form a ═CH₂ group.

2. The process according to claim 1, which further comprises reacting I with hydroxylamine VII
NH₂—OH VI
or its salt, to yield a Z-oxime of formula VI

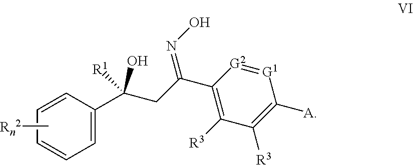

3. The process according to claim 2, wherein the solvent comprises predominantly of pyridine, 2,6-lutidine, 2,3-lutidine, 2,5-lutidine, or 2-methyl pyridine, neat or as mixture with one another.

4. The process according to claim 2, which further comprises cyclisation of formula VI compounds under basic conditions to yield a compound of formula Va

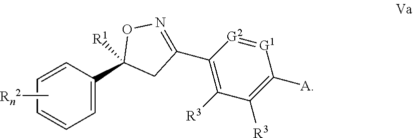

5. The process according to claim 4, which further comprises reacting Va wherein A is COOR⁹ with an amine HNR⁵R⁶ to yield a compound V wherein A is C(═O)NR⁵R⁶.

6. The process according to claim 5, wherein the compound of formula V is compound V.2

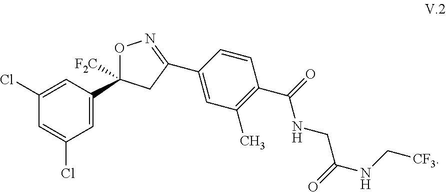

7. The process according to claim 1, wherein the catalyst of formula IV is compound IVa-1

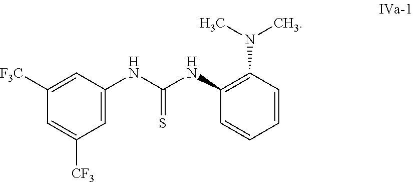

8. The process according to claim 1, wherein the catalyst of formula IV is compound IVb-1

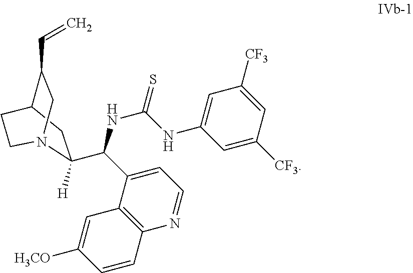 [SMILES: C=C[C@H]1CN2CCC1C[C@H]2[C@@H](NC(=S)Nc1cc(C(F)(F)F)cc(C(F)(F)F)c1)c1ccnc2ccc(OC)cc12]

9. The process according to claim 1, wherein the catalyst of formula IV is selected from compound IVb-2, IVb-3, and IVb-4

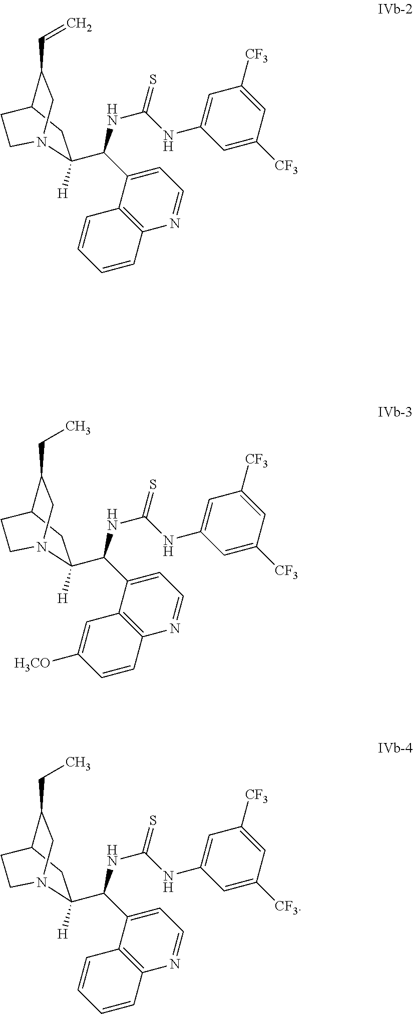 [SMILES: C=C[C@H]1CN2CCC1C[C@H]2[C@@H](NC(=S)Nc1cc(C(F)(F)F)cc(C(F)(F)F)c1)c1ccnc2ccccc12 ; CC[C@H]1CN2CCC1C[C@H]2[C@@H](NC(=S)Nc1cc(C(F)(F)F)cc(C(F)(F)F)c1)c1ccnc2ccc(OC)cc12 ; CC[C@H]1CN2CCC1C[C@H]2[C@@H](NC(=S)Nc1cc(C(F)(F)F)cc(C(F)(F)F)c1)c1ccnc2ccccc12]

10. The process according to claim 1, wherein the phenyl ring in formula I and II, bearing the R²ₙ substitution is preferably a group P 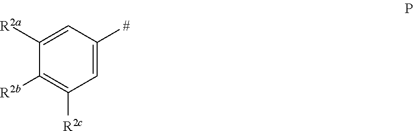
wherein R^{2a} is F, Cl, Br, CF₃, or OCF₃, and R^{2b} and R^{2c} are H, or as defined for R^{2a}.

11. The process according to claim 1, wherein G¹ is C—CH₃, or C—Cl, and G² is CH.

12. The process according to claim 1, wherein in formulae I and III, is COOR⁹, wherein R⁹ is C₁-C₄-alkyl.

13. The process according to claim 1, wherein the catalyst of formula IV is applied in 0.01 to 0.5 mol equivalents of compound II.

14. A Z-oxime of formula VI which corresponds to formula Via 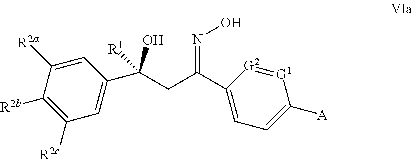
wherein
R¹ is CF₃;
R^{2a} is F, Cl, Br, CF₃, or OCF₃;
R^{2b} and R^{2c} are independently from each other FI, F, Cl, Br, CF₃, or OCF₃;
A is A¹, A², or A³; wherein
A¹ is C(═O)N(R⁵)R⁶, C(═O)OR⁹, wherein
A² is 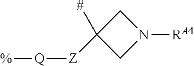
wherein # denotes the bond of group A, and % denotes the bond to G¹;
Q-Z is %-CH₂—O—*, wherein % marks the bond of Q to phenyl, and * the bond of Z to azetidin; and
R^{A4} is H, or C(═O)R^{4A}, wherein
R^{4A} is H, C₁-C₄-alkylcarbonyl, which is unsubstituted or substituted with S(O)ₙ—C₁-C₆-alkyl;
A³ is CH₂—NR⁵C(═O)R⁶;
G¹, and G² are each CR³, or together form a sulfur atom;
R³ is H or C₁-C₄-alkyl, or two R³ bonded to adjacent carbon atoms may form a five- or six-membered saturated or aromatic carbocyclic ring, or a dihydrofurane, or
R³ bonded to a carbon atom in position G¹ form a bond to the chain *-Q-Z— in group A²;
R⁵ is H;
R⁶ is H, or C₁-C₆-alkyl which is unsubstituted, or substituted with one or two R⁸;
or R⁵ and R⁶, together with the nitrogen atom to which they are bound, form a 5- or 6-membered saturated, heterocyclic ring, which ring contain 1 or 2 groups selected from O, S, N, and C═O as ring members, which heterocyclic ring is unsubstituted or partially substituted with same or different C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl;
each R⁸ is C(═O)N(R^{10a})R^{10b}, or
two R⁸ present on the same carbon atom of an alkyl group together form ═NOR⁹;
R⁹ being C₁-C₄-alkyl;
R^{10a}, R^{10b} are independently from one another H, C₁-C₆-alkyl, C₁-C₆-haloalkyl, C₂-C₆-alkenyl, C₂-C₆-haloalkenyl, C₂-C₆-alkynyl, C₂-C₆-haloalkynyl.

15. A compound of formula VI according to claim 14 wherein R¹ is CF₃, R² is as defined in claim 14, G¹ is C—CH₃ or C—Cl, G² is CH, or G¹ and G² are both C—R³, wherein two R³ bonded to adjacent carbon atoms form a five- or six-membered saturated carbocyclic ring or a dihydrofurane, and A is C(═O)Y, wherein Y is NHCH₃, or C₁-C₄-alkoxy.
